(19) United States
(12) Patent Application Publication
(10) Pub. No.: US 2012/0184493 A1
(43) Pub. Date: Jul. 19, 2012

(54) DERMAL FORMULATIONS OF DP2 RECEPTOR ANTAGONISTS

(75) Inventor: John Howard Hutchinson, San Diego, CA (US)

(73) Assignee: PANMIRA PHARMACEEUTICALS, LLC, San Diego, CA (US)

(21) Appl. No.: 13/387,047

(22) PCT Filed: Jul. 28, 2010
(86) PCT No.: PCT/US10/43599

§ 371 Date: Apr. 2, 2012

Related U.S. Application Data
(60) Provisional application No. 61/230,585, filed on Jul. 31, 2009.

(51) Int. Cl. A61K 31/195; A61K 31/437; A61K 31/403; A61K 38/13; A61P 17/00; A61P 17/02; A61P 35/00; A61P 29/00; A61P 17/06; A61P 17/12; A61P 17/04; A61P 31/00; A61P 37/08; A61P 17/08; A61P 31/04; A61P 31/12; A61P 31/10; A61P 37/00; A61K 31/56; A61K 31/4418

(52) U.S. Cl. 514/18.6; 514/564; 514/562; 514/357; 514/292; 514/411; 514/563; 514/346; 514/171

(57) ABSTRACT

Described herein are topical formulations for use in the treatment or prevention of dermatological diseases, disorders, or conditions in a mammal. Topical formulations disclosed herein include a DP₂ receptor antagonist compound formulated for dermal administration

### RELATED APPLICATIONS

This application claims the benefit of U.S. provisional patent application No. 61/230,585 entitled “DERMAL FORMULATIONS OF DP₂ RECEPTOR ANTAGONISTS” filed on Jul. 31, 2009, which is incorporated by reference in its entirety.

### FIELD OF THE INVENTION

Described herein are pharmaceutical compositions for topical administration to the skin of a mammal that include at least one DP₂ receptor antagonist compound and methods of use thereof in the treatment or prevention of dermal diseases or conditions.

### BACKGROUND OF THE INVENTION

Dermal diseases or conditions include, but are not limited to, dermatitis, psoriasis, eczema, urticaria, rosacea, burns, scarring and cutaneous mucinoses. In certain instances, dermal conditions result from an over-production of prostaglandin D₂ and/or cytokines

### SUMMARY OF THE INVENTION

Prostaglandins have a diverse range of activities and have a well recognized role in inflammation. Prostaglandin D₂ (PGD₂) is produced by mast cells, macrophages and Th2 lymphocytes in response to local tissue damage and/or inflammation and/or infection related to dermal diseases or conditions. PGD₂ binds to a number of receptors, which include the thromboxane-type prostanoid (TP) receptor, PGD₂ receptor (DP, also known as DP₁) and chemoattractant receptor-homologous molecule expressed on Th2 cells (CRTH2; also known as DP₂). In Th2 lymphocytes, IL-4, IL-5 and IL-13 cytokine production is stimulated. These cytokines have been implicated in numerous biological actions including, by way of example only, inflammation, and/or eosinophil recruitment. Topical administration of a DP₂ receptor antagonist compound is used to treat or prevent dermal diseases or conditions. Topical dermal formulations of DP₂ receptor antagonists, administered to any dermal site of a mammal, are used to prevent, ameliorate or treat DP₂-dependent or DP₂-mediated diseases or conditions.

Described herein, in certain embodiments, are topical formulations for treating a dermatological disease or condition (i.e., an abnormal state of the epidermis, dermis, and/or subcutaneous tissues). Described herein, in certain embodiments, are topical formulations for treating or preventing dermal immune diseases or conditions (e.g. autoimmune diseases or conditions (e.g., eczema, psoriasis)); dermal proliferative diseases or conditions (e.g., melanoma); contact with an allergen, and/or an irritant; scarring; a burn (e.g., first degree, second degree, third degree, or fourth degree); cutaneous mucinoses; dermal inflammatory diseases or conditions or combinations thereof. In some embodiments, a topical formulation disclosed herein comprises a therapeutically-effective amount of a DP₂ receptor antagonist compound. In some embodiments, a topical formulation disclosed herein is administered before or after contact with an allergen and/or irritant. In some embodiments, a topical formulation disclosed herein is administered before or after a physical trauma (e.g., surgery).

Provided herein, in some embodiments, is a topical formulation comprising a DP₂ receptor antagonist in an amount effective for the treatment of a dermal disease or condition, and at least one pharmaceutically acceptable excipient to provide an ointment, cream, lotion, paste, gel, stick, a film, a patch or wound dressing, wherein the topical formulation is suitable for administration to the skin of a mammal.

Provided herein, in some embodiments, is a topical formulation comprising a DP₂ receptor antagonist in an amount effective for antagonizing dermal DP₂ receptors, and at least one suitable pharmaceutically acceptable excipient to provide an ointment, cream, lotion, paste, gel, stick, a film, a patch or wound dressing.

In some embodiments, the dermal disease or condition is scarring, dermatitis, a proliferative disease or condition, a mast cell diseases or conditions, a burn, contact with an allergen and/or an irritant, or an inflammatory disease or condition. In some embodiments, the dermal disease or condition is atopic dermatitis, bullous disorders, collagenoses, psoriasis, psoriatic lesions, contact dermatitis, eczema, urticaria, rosacea, hypertrophic scarring, keloid scar formation, scleroderma, Folliculitis keloidalis nuchae, Kawasaki Disease, Sjogren-Larsso Syndrome, Grover's disease, a first degree burn, a second degree burn, a third degree burn, a fourth degree burn, cutaneous mucinosis, solar keratosis, squamous cell carcinoma or melanoma.

In some embodiments, the dermal disease or condition is dermatitis. In some embodiments, the dermal disease or condition is eczema. In some embodiments, the dermal disease or condition is uticaria. In some embodiments, the dermal disease or condition is psoriasis.

In some embodiments, the dermal disease or condition results from surgery. In some embodiments, the dermal disease or condition is cutaneous mucinosis.

In some embodiments, the DP₂ receptor antagonist is a compound of Formula (I), Formula (II), Formula (III), or Formula (IV), or a pharmaceutically acceptable salt, pharmaceutically acceptable solvate, metabolite, or prodrug thereof. In some embodiments, the DP₂ receptor antagonist is a compound of Formula (I), or a pharmaceutically acceptable salt, pharmaceutically acceptable solvate, metabolite, or prodrug thereof. In some embodiments, the DP₂ receptor antagonist is a DP₂ receptor antagonist disclosed herein.

In some embodiments, a topical formulation described herein further comprises a second therapeutic agent. In some embodiments, the second therapeutic agent is an antibiotic, anti-fungal agent, steroid anti-inflammatory agent, non-steroidal anti-inflammatory agent, antihistamine, antiviral agent, mast cell stabilizer, cyclosporine, or a leukotriene modulator. In some embodiments, the second therapeutic agent is a leukotriene modulator selected from 5-lipoxygenase (5-LO) inhibitors, 5-lipoxygenase activating protein (FLAP) inhibitors, and leukotriene receptor antagonists.

Also provided herein is a method of treating a prostaglandin D₂-dependent or prostaglandin D₂-mediated dermal disease or condition, comprising administering to a mammal in need thereof a therapeutically-effective amount of a topical formulation described above.

Also provided herein is a method of antagonizing dermal DP₂ receptors in a mammal in need thereof, comprising administering to the mammal a therapeutically-effective amount of a topical formulation described above.

In some embodiments of the methods, the prostaglandin D₂-dependent or prostaglandin D₂-mediated dermal disease or condition is scarring, dermatitis, a proliferative disease or condition, a mast cell disease or condition, a burn, contact with an allergen and/or an irritant, or an inflammatory disease or condition.

In some embodiments of the methods, the prostaglandin D₂-dependent or prostaglandin D₂-mediated dermal disease or condition is atopic dermatitis, bullous disorders, collagenoses, psoriasis, psoriatic lesions, contact dermatitis, eczema, urticaria, rosacea, hypertrophic scarring, keloid scar formation, scleroderma, Folliculitis keloidalis nuchae, Kawasaki Disease, Sjogren-Larsso Syndrome, Grover's disease, a first degree burn, a second degree burn, a third degree burn, a fourth degree burn, cutaneous mucinosis, solar keratosis, squamous cell carcinoma or melanoma.

In some embodiments of the methods, the prostaglandin D₂-dependent or prostaglandin D₂-mediated dermal disease or condition is dermatitis. In some embodiments of the methods, the prostaglandin D₂-dependent or prostaglandin D₂-mediated dermal disease or condition is atopic dermatitis or allergic dermatitis. In some embodiments of the methods, the prostaglandin D₂-dependent or prostaglandin D₂-mediated dermal disease or condition is eczema. In some embodiments of the methods, the prostaglandin D₂-dependent or prostaglandin D₂-mediated dermal disease or condition is uticaria. In some embodiments of the methods, the prostaglandin D₂-dependent or prostaglandin D₂-mediated dermal disease or condition is psoriasis. In some embodiments of the methods, the prostaglandin D₂-dependent or prostaglandin D₂-mediated dermal disease or condition results from surgery. In some embodiments of the methods, the prostaglandin D₂-dependent or prostaglandin D₂-mediated dermal disease or condition is cutaneous mucinosis.

Also provided herein is a method of increasing the dermal concentration of a DP₂ receptor antagonist compound in a mammal comprising administering to a mammal in need thereof a therapeutically effective amount of a topical formulation described herein. In some embodiments of the method, the mammal has at least one symptom of a prostaglandin D₂-dependent or prostaglandin D₂-mediated dermal disease or condition. In some embodiments of the method, the dermatological disease or condition results from surgery. In some embodiments of the method, the topical formulation is administered before surgery. In some embodiments of the method, the topical formulation is administered after surgery. In some embodiments of the method, the topical formulation is administered before contact with an irritant and/or allergen. In some embodiments of the method, the topical formulation is administered after contact with an irritant and/or allergen.

In one aspect, provided is a method for the treatment or prevention of itching in a mammal comprising administering to the mammal a therapeutically-effective amount of a topical formulation described herein comprising a DP2 receptor antagonist. In some embodiments, the itching is a symptom of any of the diseases or conditions described herein. In some embodiments, the itching is a symptom of any of the PGD₂-dependent or PGD₂-mediated diseases or conditions described herein. In some embodiments, the itching is caused by contact with an irritant, allergen, or combination thereof. In some embodiments, the itching is a symptom of dermatitis, eczema, urticaria, or psoriasis. In some embodiments, the itching is a symptom of atopic dermatitis or allergic dermatitis.

In one aspect, provided herein is a method for the treatment or prevention of a rash in a mammal comprising administering to the mammal a therapeutically-effective amount of a topical formulation described herein comprising a DP2 receptor antagonist. In some embodiments, the rash is a symptom of any of the diseases or conditions described herein. In some embodiments, the rash is a symptom of any of the PGD₂-dependent or PGD₂-mediated diseases or conditions described herein. In some embodiments, the rash is caused by contact with an irritant, allergen, or combination thereof. In some embodiments, the rash is a symptom of dermatitis, eczema, urticaria, or psoriasis. In some embodiments, the rash is a symptom of atopic dermatitis or allergic dermatitis.

In one aspect, provided herein is a method for the treatment or prevention of skin inflammation in a mammal comprising administering to the mammal a therapeutically-effective amount of a topical formulation described herein comprising a DP2 receptor antagonist. In some embodiments, the skin inflammation is a symptom of any of the diseases or conditions described herein. In some embodiments, the skin inflammation is a symptom of any of the PGD₂-dependent or PGD₂-mediated diseases or conditions described herein. In some embodiments, the skin inflammation is caused by contact with an irritant, allergen, or combination thereof. In some embodiments, the skin inflammation is a symptom of dermatitis, eczema, urticaria, or psoriasis. In some embodiments, the skin inflammation is a symptom of atopic dermatitis or allergic dermatitis.

In one aspect, provided herein is a method for the treatment or prevention of blisters, redness, swelling, scabbing, scaling, or combinations thereof in a mammal comprising administering to the mammal a therapeutically-effective amount of a topical formulation described herein comprising a DP2 receptor antagonist. In some embodiments, the blisters, redness, swelling, scabbing, scaling, or combinations thereof is a symptom of any of the diseases or conditions described herein. In some embodiments, the blisters, redness, swelling, scabbing, scaling, or combinations thereof is a symptom of any of the PGD₂-dependent or PGD₂-mediated diseases or conditions described herein. In some embodiments, the blisters, redness, swelling, scabbing, scaling, or combinations thereof is caused by contact with an irritant, allergen, or combination thereof. In some embodiments, the blisters, redness, swelling, scabbing, scaling, or combinations thereof is a symptom of dermatitis, eczema, urticaria, or psoriasis. In some embodiments, the blisters, redness, swelling, scabbing, scaling, or combinations thereof is a symptom of atopic dermatitis or allergic dermatitis.

In one aspect is the use of a DP₂ receptor antagonist compound in the manufacture of a topical formulation for application to the skin. In one aspect is the use of a combination of a DP₂ receptor antagonist compound and a second therapeutic agent (e.g. a FLAP inhibitor compound) in the manufacture of a topical formulation for application to the skin.

In one aspect is the use of a DP₂ receptor antagonist compound in the treatment or prevention of a dermal disease or condition in a mammal. In one aspect is the use of a DP₂ receptor antagonist compound in the treatment or prevention of a dermal disease or condition in a mammal, wherein the DP₂ receptor antagonist compound is in a form suitable for topical administration to the skin of a mammal.

In one aspect is the use of DP₂ receptor antagonist compound in the manufacture of a topical formulation for the treatment of a dermal disease or condition. In one aspect is the use of a DP₂ receptor antagonist compound and a second therapeutic agent (e.g., a FLAP inhibitor compound) in the manufacture of a topical formulation for the treatment of a dermal disease or condition.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description

### BRIEF DESCRIPTION OF THE FIGURES

FIG. 1 illustrates the effect of DP₂ receptor antagonism, alone or in combination with FLAP inhibition, on the number of total cells, neutrophils and lymhocytes present in BALF in a mouse subchronic smoke model

FIG. 2 illustrates the effect of a DP₂ receptor antagonist, alone or in combination with a FLAP inhibitor, on the presence of mucin in BALF

### DETAILED DESCRIPTION OF THE INVENTION

Prostaglandin D₂ (PGD₂) is an acidic lipid derived from the metabolism of arachidonic acid by cyclooxygenases and PGD₂ synthases. PGD₂ is produced by mast cells, macrophages and Th2 lymphocytes in response to local tissue damage as well as in response allergic inflammation and/or infection. PGD₂ exerts a variety of biologic actions in the skin; these include scarring, mucinosis and inflammatory effects.

Activation of DP₂ is associated with chemotaxis and activation of Th2 lymphocytes, eosinophils and basophils. PGD₂ binds to DP₂ receptors and mediates many of its effects through a Gᵢ-dependent elevation of intracellular calcium levels and reduction of cyclic AMP. In Th2 lymphocytes, IL-4, IL-5 and IL-13 cytokine production are also stimulated by DP₂ receptor activation. These cytokines have been implicated in numerous biological actions including, by way of example only, immunoglobulin E production, mucous secretion and/or accumulation, and eosinophil recruitment.

DP₂ receptors provide a target for the treatment of PGD₂-dependent or PGD₂-mediated dermal diseases, disorders or conditions, including, by way of example, immune diseases or conditions, (e.g. an autoimmune diseases or conditions (e.g., eczema, psoriasis)); proliferative conditions (e.g., melanoma); contact with an allergen and/or an irritant; a mast cell diseases or conditions; scarring (e.g., scarring after a trauma (e.g., surgery)); burns; cutaneous mucinosis; inflammatory diseases or conditions affecting the skin, or combinations thereof.

Disclosed herein is the use of DP₂ receptor antagonist compounds in the manufacture of medicaments suitable for topical administration to the skin of a mammal for use in the treatment or prevention of prostaglandin D₂-dependent or prostaglandin D₂-mediated dermal diseases or conditions.

Described herein are pharmaceutical compositions suitable for topical administration, methods for treating, methods for formulating topical formulations, methods for producing, methods for manufacturing, and treatment strategies using DP₂ receptor antagonist compounds.

Described herein, in certain embodiments, are topical formulations that include a DP₂ receptor antagonist compound for treating or preventing a dermatological disease or condition. In one aspect, topical administration of a DP₂ receptor antagonist compound to a mammal minimizes systemic absorption of the DP₂ receptor antagonist compound. In one aspect, topical administration of a DP₂ receptor antagonist compound allows for local treatment of dermal conditions. In one aspect, local treatment of dermal conditions with a DP₂ receptor antagonist compound reduces possible side effects associated with systemic administration of a DP₂ receptor antagonist compound.

In one aspect, the dermatological condition is a result of the over-production of PGD₂ and/or cytokines In one aspect, the dermatological disease or condition includes, but is not limited to, dermatological immune diseases or conditions, dermatological proliferative conditions, a dermatological disease or condition resulting from contact with an allergen and/or an irritant, a dermatological mast cell diseases or conditions, a burn, a cutaneous mucinosis, an inflammatory disease or condition affecting the skin, or combinations thereof. Allergens and/or irritants include, but are not limited to, uruishol, alcohol, xylene, turpentine, esters, acetone, ketones. Dermatological immune disorders include, but are not limited to, eczema, psoriasis. Dermatological proliferative disorders include, but are not limited to, melanoma. Dermatological mast cell disorders include but are not limited to, fibroblast disorders including scarring, such as the formation of keloid scars, hypertrophic scars, and/or acne scars. Dermatological burn disorders include, but are not limited to, a first degree burn, a second degree burn, a third degree burn, or a fourth degree burn.

Described herein, in certain embodiments, are topical formulations that include a DP₂ receptor antagonist compound for treating a chronic blistering disorder, psoriasis, dermatitis (e.g., contact or atopic), eczema, urticaria, rosacea, scarring (i.e. the formation of a scar (e.g., a keloid scar or a hypertrophic scar)), a first degree burn, a second degree burn, a third degree burn, a fourth degree burn, cutaneous mucinosis and/or melanoma. In some embodiments, a topical formulation disclosed herein comprises a therapeutically-effective amount of a DP₂ receptor antagonist. In some embodiments, a topical formulation disclosed herein is administered before or after contact with an allergen and/or irritant. In some embodiments, a topical formulation disclosed herein is administered before or after a physical trauma (e.g., surgery). In one aspect, a topical formulation disclosed herein that includes a DP₂ receptor antagonist compound is topically administered to treat and prevent scar formation following surgery. It is understood that the topical formulation is applied to the site of injury.

In certain instances, prostaglandin D₂ and/or cytokines are involved in scarring and/or the migration of eosinophils. In one aspect, inhibiting the activity of DP₂ receptors inhibits the activity of and/or migration of eosinophils, and/or treats scarring. In certain instances, inhibiting the activity of DP₂ receptors reduces or inhibits the deposition of mucin in the interstitial spaces of the dermis.

In one aspect, PGD₂ is involved in the pathogenesis of dermatological diseases or conditions described herein. In some instances, inhibition of binding of PGD₂ to DP₂ receptors will result in a decrease in the production of cytokines In some instances, a reduction of production of cytokines results in a decrease of inflammation and/or fibrosis and/or mucinosis.

### Dermatological Diseases or Conditions

Described herein, in certain embodiments, are topical formulations for treating a dermatological disease or condition (e.g., dermatoses). As used herein, a dermatological disease or condition includes any abnormal state of the epidermis, dermis, and/or subcutaneous tissues. In certain instances, a dermatological disease or condition is caused by an immune disease or condition, (e.g. an autoimmune disease or condition); a proliferative disease or condition; contact with an allergen and/or an irritant; a mast cell disease or condition, scarring, a burn, cutaneous mucinosis, inflammatory disease or condition, or combinations thereof. Dermatological diseases or conditions include, but are not limited to, a chronic blistering (bullous) disorder, psoriasis, dermatitis (e.g., contact or atopic), eczema, urticaria, rosacea, scarring (i.e. the formation of a scar (e.g., a keloid scar or a hypertrophic scar)), a first degree burn, a second degree burn, a third degree burn, a fourth degree burn, cutaneous mucinosis and/or melanoma.

In some embodiments, a topical formulation disclosed herein is administered before or after contact with an allergen and/or irritant.

In some embodiments, treating or preventing any of the diseases or conditions described herein reduces the severity of or prevents the occurence of at least one symptom of the disease or condition. In some embodiments, dermatological diseases or conditions are accompanied by inflammation of the upper layers of the skin. In some embodiments, inflammation of the upper layers of the skin causes itching, blisters, redness, swelling, oozing, scabbing, and scaling. In some embodiments, inflammation of the upper layers of the skin results in a rash, blisters, pimples, open sores, oozing, crusting, and scaling.

### Dermatitis

In some embodiments, a topical formulation disclosed herein is administered to treat a dermatological disease or condition, wherein the dermatological disease or condition is dermatitis. As used herein, dermatitis means an inflammatory condition of the skin. In certain instances, dermatitis is acute and results from contact with an offending agent (e.g., uruishol). In certain instances, dermatitis is chronic and results from hypersensitivity.

Types of dermatitis include, but are not limited to: spongiotic dermatitis (irritant dermatitis, seborrheic dermatitis, atopic dermatitis, allergic contact dermatitis, thermal induced dermatitis, and drug induced dermatitis); allergic contact dermatitis (contact dermatitis can be due to external compounds, preservatives, fragrances, or plants); seborrhoeic dermatitis (seborrhoeic dermatitis is also known as dandruff); dyshidrotic dermatitis (also known as Pompholyx); vesicular or bullous dermatitis (can be caused by drug reaction, or auto immune diseases; examples includes Steven Johnson Syndrome, bullous erythema multiforme, bullous pemphigoid, and pemphigus vulgaris).

In certain instances, the symptoms of dermatitis (e.g., chronic or acute) result from a disorder of an immune system. In certain instances, the symptoms of dermatitis (e.g., chronic or acute) result from the exudation of plasma from vessels and capillaries into the epidermis, dermis, and/or subcutaneous tissues. In certain instances, cytokines cause inflammation associated with dermatitis. In certain instances, inhibiting DP₂ receptor activity reduces the concentration of cytokines associated with dermatitis. In certain instances, inhibiting DP₂ receptor activity reduces exudation of plasma from vessels and capillaries associated with dermatitis. In certain instances, antagonism of DP₂ receptors treats dermatitis.

In some embodiments, dermatitis is atopic dermatitis or allergic dermatitis. In some embodiments, dermatitis is atopic dermatitis. In some embodiments, dermatitis is allergic dermatitis.

### Psoriasis

In some embodiments, a topical formulation disclosed herein is administered to treat a dermatological disease or condition, wherein the dermatological disease or condition is psoriasis. In certain instances, the symptoms of psoriasis result from influx of cytokines into the epidermis, dermis, and/or subcutaneous tissues. In certain instances, cytokines cause inflammation and subsequent psoriasis. In certain instances, inhibiting DP₂ activity reduces the concentration of cytokines associated with psoriasis. In certain instances, antagonism of DP₂ receptors treats psoriasis.

### Eczema

In some embodiments, a topical formulation disclosed herein is administered to treat a dermatological disease or condition, wherein the dermatological disease or condition is eczema. As used herein, eczema is an inflammation of the epidermis. The symptoms of this persistent skin condition include dryness and recurring skin rashes which are characterized by one or more of inflammation (e.g., redness), skin edema (swelling), itching and dryness, crusting, flaking, blistering, cracking, oozing, or bleeding. In certain instances, inhibiting DP₂ receptor activity reduces the concentration of cytokines associated with eczema. In certain instances, inhibiting DP₂ receptor activity reduces inflammation associated with eczema. In certain instances, antagonism of DP₂ receptors treats eczema.

### Urticaria

In some embodiments, a topical formulation disclosed herein is administered to treat a dermatological disease or condition, wherein the dermatological disease or condition is urticaria. In certain instances, urticaria results from hypersensitivity or another immune disorder. In certain instances, the symptoms of urticaria result from influx of cytokines into the epidermis, dermis, and/or subcutaneous tissues. In certain instances, cytokines cause the inflammation and/or hypersensitivity associated with urticaria. In certain instances, inhibiting DP₂ receptor activity reduces the concentration of cytokines associated with uticaria. In certain instances, antagonism of DP₂ receptors treats urticaria.

In some embodiments, the uticaria is papular urticaria.

### Type I Hypersensitivity

In some embodiments, topical formulations disclosed herein are administered to mammal to treat or prevent type I hypersensitivity (or immediate hypersensitivity). Type I hypersensitivity is an allergic reaction provoked by exposure to an irritant or allergen or combination thereof. Exposure may be by ingestion, inhalation, injection, or direct contact. Non-limiting examples of type I hypersensitivity include allergic dermatitis, urticaria, and food allergy.

### Itch

In some embodiments, a topical formulation disclosed herein is administered to treat or prevent itching in a mammal. In some embodiments, the itching is a symptom of any of the diseases or conditions disclosed herein. In some embodiments, the itching is a result of contact with an irritant, allergen, or combination thereof. In some embodiments, the itching is a result of the PGD₂-dependent or PGD₂-mediated diseases or conditions that are disclosed herein (e.g. atopic dermatitis, allergic contact dermatitis, urticaria, and the like). In some embodiments, the topical formulations disclosed herein reduce itching that is associated with contact with an irritant, allergen, or combination thereof. In some embodiments, the topical formulations disclosed herein reduce itching that is associated with dermatitis, psoriasis or uticaria.

### Bullous Diseases or Conditions

In some embodiments, a topical formulation disclosed herein is administered to treat a dermatological disease or condition, wherein the dermatological disease or condition is a bullous disease or condition. In certain instances, a bullous disease or condition is characterized by the formation of blisters (i.e., the accumulation of fluid between cells in the upper layers of the skin). In certain instances, bullous disease or condition are an immune disease or condition. In certain instances, PGD₂ and/or cytokines mediate the formation of blisters (e.g., induce the exudation of plasma from capillaries to the upper layers of the skin). In certain instances, inhibiting DP₂ receptor activity reduces the concentration of cytokines associated with bullous disease or condition, and, further, treats bullous diseases or conditions. Bullous diseases or conditions include, but are not limited to, bullous pemphigoid, pemphigus vulgaris, pemphigus vegetans, pemphigus foliaceous, paraneoplastic pemphigus, mucous membrane pemphigoid, linear IgA bullous disease, dermatitis herpeti-formis, and epidermolysis bullosa acquisita.

### Rosacea

In some embodiments, a topical formulation disclosed herein is administered to treat a dermatological disease or condition, wherein the dermatological disease or condition is rosacea. As used herein, rosacea refers to any of erythematotelangiectatic rosacea (ETR), Papulopustular rosacea, and/or Phymatous rosacea. In certain instances, inhibiting or reducing the binding of PGD₂ to DP₂ receptors treats rosacea.

### Skin Ulcers

In some embodiments, a topical formulation disclosed herein is administered to treat a dermatological disease or condition, wherein the dermatological disease or condition is skin ulcers. As used herein, an ulcer is a disease or condition of the skin characterized by degradation of the epidermis and often portions of the dermis and even subcutaneous fat. In certain instances, ulcers are areas of necrotic tissue. In certain instances, ulcers result from immune system dysfunction. In certain instances, ulcers result from immune system dysfunction such as, but not limited to, the improper functioning of neutrophils. In certain instances, PGD₂ and/or cytokines are chemotactic agents for eosinophils. In certain instances, inhibiting DP₂ receptor activity reduces the concentration of cytokines associated with skin ulcers. In certain instances, inhibiting DP₂ receptor activity reduces the chemotaxis of eosinophils associated with skin ulcers. In certain instances, antagonism of DP₂ receptors treats skin ulcers.

### Scarring

In some embodiments, a topical formulation disclosed herein is administered to treat a dermatological disease or condition, wherein the dermatological disease or condition is scarring. As used herein, scarring refers to the formation of a scar. In one aspect, the scar is a hypertrophic scar, or keloid scar, or a scar resulting from acne. In certain instances, a scar is an area of fibrous tissue that results from inflammation (e.g., the overproduction of cytokines and/or collagen). In certain instances a scar is a result of an infection (e.g. acne). In certain instances, cytokines modulate the inflammation associated with scarring. In certain instances, inhibiting the activity of DP₂ receptors reduces or inhibits the activity of mast cells and/or over-production of cytokines associated with scarring. In certain instances, inhibiting DP₂ receptors activity reduces the concentration of cytokines associated with scarring. In certain instances, antagonism of DP₂ receptors treats scarring.

### Burns

In some embodiments, a topical formulation disclosed herein is administered to treat a dermatological disease or condition, wherein the dermatological disease or condition is a burn. As used herein, a burn refers to an injury to or the destruction of skin caused by heat, cold, electricity, chemicals, light (e.g. a sunburn caused by UV exposure), radiation, or friction. In one aspect, the burn is a first degree burn, a second degree burn, a third degree burn, or a fourth degree burn. In certain instances, a burn results in the formation of a scar. In certain instances, a burn results in inflammation. In certain instances, inhibiting the activity of DP₂ receptors inhibits the activity of mast cells and/or eosinophils associated with scarring and/or inflammation. In certain instances, inhibiting the binding of PGD₂ to DP₂ receptors reduces the concentration of cytokines associated with scarring and/or inflammation. In certain instances, inhibiting DP₂ receptor activity treats scarring and/or inflammation associated with burns.

### Cutaneous Mucinoses

In some embodiments, a topical formulation disclosed herein is administered to treat a dermatological disease or condition, wherein the dermatological disease or condition is cutaneous mucinosis. Cutaneous mucinosis refers to diseases or conditions wherein mucin accumulates in the dermis. In some embodiments, accumulation of mucin occurs in the interstitial spaces of the dermis and within hair follicles. In certain instances, mucinosis results from a mast cell disorder. PGD₂ and/or cytokines released by mast cells upregulate the synthesis of mucin from fibroblasts. In certain instances, deposition of mucin causes papules or nodules within or outside skin lesions (e.g., in skin lesions associated with systemic lupus erythematosus, discoid lupus erythematosus, and/or subacute cutaneous lupus erythematosus). In certain instances, inhibiting the activity of DP₂ receptors inhibits the activity of mast cells and/or fibroblasts associated with deposition of mucin and/or mucinoses. In certain instances, inhibiting the binding of PGD₂ to DP₂ receptors reduces the concentration of cytokines associated with mucinoses. In certain instances, inhibiting DP₂ receptor activity treats or prevents cutaneous mucinoses.

Cutaneous mucinoses are observed in diseases or conditions such as, but are not limited to, generalized myxedema, pretibial myxedema, reticular erythematous mucinosis, scleredema, scleromyxedema, papular mucinosis, acral persistent papular mucinosis, focal mucinosis, digital mucous cyst, mucocele, cutaneous myxoma, cutaneous mucinosis of infancy, nevus mucinosis, alopecia mucinosa (follicular mucinosis), mucopolysaccharidoses, Degos disease, dermatomyositis, granuloma annulare, Jessner's lymphocytic infiltrate, lupus erythematosus, papulonodular mucinosis associated with systemic lupus erythematosus (SLE), lichen-myxedematosus, dysthyroidotic mucinoses (mucinoses associated with thyroid diseases), reticular erythematous mucinosis, acral persistent mucinosis, juvenile cutaneous mucinosis, cutaneous mucinosis of infancy, cutaneous toxic-oil mucinoses, neuropathy-related cutaneous mucinosis, urticaria-like mucinosis, hereditary mucinous histiocytosis and primary mucinous skin (sebaceous and sweat glands) carcinomas.

In some embodiments, any topical formulation described herein comprises a DP₂ receptor antagonist in combination with a second therapeutic agent (e.g., leukotriene modulator such as a FLAP inhibitor compound) and is administered to the skin for the treatment of any prostaglandin D₂-mediated or prostaglandin D₂-dependent disease or condition described herein. In some embodiments, the effects of a DP₂ receptor antagonist compound and the second therapeutic agent are additive, i.e., administration of a combination of a DP₂ receptor antagonist compound and the second therapeutic agent provides greater therapeutic benefit than administration of either compound alone.

### DP₂ Receptor Antagonists

In one aspect, the pharmaceutical compositions disclosed herein comprise at least one DP₂ receptor antagonist compound. In some embodiments, the DP₂ receptor antagonist is selected from compounds disclosed in International patent application no. PCT/US09/35174 (entitled Antagonists of Prostaglandin D₂ receptors); International patent application no. PCT/US08/82056 (entitled Antagonists of PG D₂ receptors (entitled Antagonists of PG D₂ receptors“); International patent application no. PCT/US08/82082 (entitled Antagonists of PG D₂ receptors (entitled Antagonists of PG D₂ receptors”); International patent application no. PCT/US0932495 (entitled N,N-disubstituted aminoalkylbiphenyl antagonists of prostaglandin D₂ receptors); International patent application no. PCT/US09/32499 (entitled “N,N-disubstituted aminoalkylbiphenyl antagonists of prostaglandin D₂ receptors”); International patent application no. PCT/US09/33961 (entitled “Cyclic diaryl ether compounds as antagonists of prostaglandin D₂ receptors”); International patent application no. PCT/US09/38291 (entitled “Aminoalkylphenyl antagonists of prostaglandin D₂ receptors”); U.S. provisional application No. 61/078,311 (entitled “Heteroalkyl antagonists of prostaglandin D₂ receptors”); U.S. provisional application No. 61/101,074 (entitled “Heteroaryl antagonists of prostaglandin D₂ receptors”); U.S. provisional application No. 61/054,093 (entitled “Tricyclic antagonists of prostaglandin D₂ receptors”); U.S. provisional application No. 61/107,638 (entitled “Tricyclic antagonists of prostaglandin D₂ receptors”); U.S. provisional application No. 61/075,242 (entitled “Cycloaklane[B]indole antagonists of prostaglandin D₂ receptors”); U.S. provisional application No. 61/101,964 (entitled “Heteroaryl antagonists of prostaglandin D₂ receptors”); U.S. provisional application No. 61/103,872 (entitled “Heteroalkyl biphenyl antagonists of prostaglandin D₂ receptors”); U.S. provisional application No. 61/115,259 (entitled “Heterocyclic antagonists of prostaglandin D₂ receptors”); U.S. provisional application No. 61/112,044 (entitled “Cycloaklane[B]azaindole antagonists of prostaglandin D2 receptors”); U.S. provisional application No. 61/147,437 (entitled “Indolozine compounds as prostaglandin D₂ receptor antagonists”); U.S. provisional application No. 61/025,597; U.S. provisional application No. 61/110,496; U.S. application No. 12/362,439; International patent application No. PCT/US09/49621; International patent application No. PCT/US09/49631; U.S. application Ser. No. 12/497,343; International patent application No. PCT/US09/58655; International patent application No. PCT/US09/58663; U.S. application Ser. No. 12/568,571; International patent application No. PCT/US09/44219; International patent application no. PCT/US09/48327; International patent application No. PCT/US09/59256; International patent application No. PCT/US09/59891; International patent application no. PCT/US09/64630; International patent application no. PCT/US09/63439; International patent application no. PCT/US09/63438; U.S. application Ser. No. 12/613,424; International patent application no. PCT/US2010/22145; or pharmaceutically acceptable salt; pharmaceutically acceptable solvate; metabolite, prodrug or N-oxide thereof, each disclosure is incorporated by reference for such compounds.

In some embodiments, the DP₂ receptor antagonist is ramatroban, AMG 009, AMG 853, Compound 14 of WO 09/085177, AZD1981, AZD8075, AZD5985, ARRY-005, ARRY-006, ARRY-063, ODC9101 (OC459), OC499, OC1768, OC2125, OC2184, QAV680, MLN6095, ACT-129968, ADC3680, SAR398171, S555739, AP768, [2′-(3-Benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxyphenyl}-acetic acid, TM30642, TM30643, TM30089, TM27632, and TM3170, {2′-[(N-cyclopropanecarbonyl-N-ethyl-amino)-methyl]-6-methoxy-4′-trifluoromethyl-biphenyl-3-yl}-acetic acid, [2′-[(N-cyclopropanecarbonyl-N-ethyl-amino)-methyl]-4′-(6-ethoxypyridin-3-yl)-6-methoxy-biphenyl-3-yl]-acetic acid, (5-{2-[(N-benzyloxycarbonyl-N-ethylamino)-methyl]-4-trifluoromethyl-phenyl}-pyridin-3-yl)-acetic acid, or {8-[(4-fluorobenzenesulfonyl)-methyl-amino]-6,7,8,9-tetrahydro-pyrido[3,2-b]indol-5-yl}-acetic acid, or pharmaceutically acceptable salt; pharmaceutically acceptable solvate; metabolite, prodrug or N-oxide thereof.

In some embodiments, the DP₂ receptor antagonist is a compound having the structure of Formula (I), pharmaceutically acceptable salt, pharmaceutically acceptable solvates, or prodrug thereof:

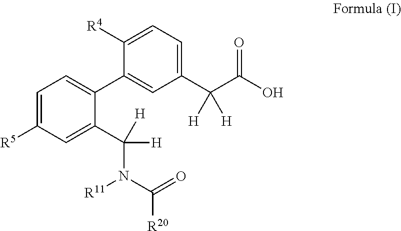

wherein,
R⁴ is H, halogen, —CN, —OH, C₁-C₄alkyl, C₁-C₄alkoxy, C₁-C₄fluoroalkyl, C₁-C₄fluoroalkoxy, or C₁-C₄heteroalkyl;
R⁵ is H, halogen, —CN, —NO₂, —OH, —OR¹³, —SR¹², —S(═O)R¹², —S(═O)₂R¹², —NHS(═O)₂R¹², —C(═O)R¹², —OC(═O)R¹², —CO₂R¹³, —OCO₂R¹³, —CH(R¹³)₂, —N(R¹³)₂, —N(R¹³)₂, —C(═O)N(R¹³)₂, —OC(═O)N(R¹³)₂, —NHC(═O)NH(R¹³), —NHC(═O)R¹², —NHC(═O)OR¹², —C(OH)(R¹³)₂, —C₁-C₆alkyl, C₁-C₆fluoroalkyl, C₁-C₆fluoroalkoxy, C₁-C₆alkoxy, or C₁-C₆heteroalkyl;
or R⁵ is C₃-C₁₀cycloalkyl, a substituted or unsubstituted C₂-C₁₀heterocycloalkyl, a substituted or unsubstituted phenyl, a substituted or unsubstituted naphthyl, a substituted or unsubstituted monocyclic heteroaryl, or a substituted or unsubstituted bicyclic heteroaryl, wherein if R⁵ is substituted, then R⁵ is substituted with 1, or 2 R²¹ groups
R²⁰ is C₁-C₄alkyl, C₃-C₆cycloalkyl, —CH₂O—C₁-C₄alkyl, —CH₂O-(substituted or unsubstituted phenyl), —CH(CH₃)—O-(substituted or unsubstituted phenyl), —C(CH₃)₂—O-(substituted or unsubstituted phenyl), —CH₂OCH₂-(substituted or unsubstituted phenyl), —OC₁-C₄alkyl, —O—CH₂-(substituted or unsubstituted phenyl), —O—CH(CH₃)-(substituted or unsubstituted phenyl), —NR¹⁶C₁-C₄alkyl, —NR¹⁶—CH₂-(substituted or unsubstituted phenyl), or —NR¹⁶—CH(CH₃)-(substituted or unsubstituted phenyl), wherein if the phenyl of R²⁰ is substituted, then the phenyl is substituted with 1, or 2 R²¹ groups;
each R²¹ is independently selected from halogen, —OH, —OC₁—C₄alkyl, C₁-C₄alkyl, and —CF₃;
R¹⁶ is H or C₁-C₄alkyl;
R¹¹ is C₁-C₄alkyl, C₁-C₄fluoroalkyl, or C₃-C₆cycloalkyl;
R¹² is C₁-C₄alkyl, C₁-C₄heteroalkyl, or C₁-C₄fluoroalkyl;
each R¹³ is independently selected from H, C₁-C₄alkyl, C₁-C₄heteroalkyl, and C₁-C₄fluoroalkyl.

In some embodiments, R¹¹ is —CH₃, —CH₂CH₃, —CF₃, —CH₂CF₃, cyclopropyl, cyclobutyl, or cyclopentyl. In some embodiments, R¹¹ is —CH₂CH₃ or —CH₂CF₃. In some embodiments, R¹¹ is —CH₂CH₃.

In some embodiments, R⁵ is H, halogen, —CN, —NO₂, —OH, —OR¹³, —SR¹², —S(═O)R¹², —S(═O)₂R¹², —NHS(═O)₂R¹², —C(═O)R¹², —OC(═O)R¹², —CO₂R¹³, —OCO₂R¹³, —N(R¹³)₂, —C(═O)N(R¹³)₂, —OC(═O)N(R¹³)₂, —NHC(═O)NH(R¹³), —NHC(═O)R¹², —NHC(═O)OR¹², —C(OH)(R¹³)₂, —C₁-C₄alkyl, C₁-C₄fluoroalkyl, C₁-C₄fluoroalkoxy, C₁-C₄alkoxy, or C₁-C₄heteroalkyl; R¹² is C₁-C₄alkyl; each R¹³ is independently selected from H, and C₁-C₄alkyl. In some embodiments, R⁵ is H, —CF₃, —CO₂H, Br, —NH—C(═O)—CH₃, —NH—C(═O)—OCH₃, —NH—SO₂CH₃, —SCH₃, —SO₂CH₃, —NH—(C═O)—CH₃, —NH—SO₂—CH₃, or —C(CH₃)₂—(OH).

In some embodiments, R²⁰ is C₁-C₄alkyl, C₃-C₆cycloalkyl, —CH₂O—C₁-C₄alkyl, —CH₂O-(substituted or unsubstituted phenyl), —CH(CH₃)—O-(substituted or unsubstituted phenyl), —C(CH₃)₂—O-(substituted or unsubstituted phenyl), —CH₂OCH₂-(substituted or unsubstituted phenyl), —OC₁-C₄alkyl, —O—CH₂-(substituted or unsubstituted phenyl), —O—CH(CH₃)-(substituted or unsubstituted phenyl), —NR¹⁶C₁-C₄alkyl, —NR¹⁶—CH₂-(substituted or unsubstituted phenyl), or —NR¹⁶—CH(CH₃)-(substituted or unsubstituted phenyl), wherein if the phenyl of R²⁰ is substituted then the phenyl is substituted with 1 or 2 R²¹ groups.

In some embodiments, R⁴ is H, F, Cl, Br, —OH, —CH₃, —OCH₃, —CF₃, or —OCF₃; R⁵ is H, halogen, —CN, —NO₂, —OH, —S(═O)₂CH₃, —NHS(═O)₂CH₃, —C(═O)CH₃, —OC(═O)CH₃, —CO₂H, —CO₂CH₃, —CO₂CH₂CH₃, —NH₂, —C(═O)NH₂, —NHC(═O)CH₃, —CH₃, —CF₃, —OCF₃, —OCH₃, —CH₂OH, or —C(CH₃)₂OH.

In some embodiments, R²⁰ is —CH₃, —CH₂CH₃, cyclopropyl, cyclobutyl, cyclopentyl, cyclohexyl, —CH₂OCH₃, —CH₂O-(substituted or unsubstituted phenyl), —CH(CH₃)—O-(substituted or unsubstituted phenyl), —C(CH₃)₂—O-(substituted or unsubstituted phenyl), —CH₂OCH₂-(substituted or unsubstituted phenyl), wherein if the phenyl of R²⁰ is substituted then the phenyl is substituted with 1 or 2 R²¹ groups.

In some embodiments, each R²¹ is independently selected from F, Cl, Br, —OH, —OCH₃, —OCH₂CH₃, —CH₂CH₃, —CH₃, and —CF₃. In some embodiments, each R²¹ is independently selected from F, Cl, Br, —OH, —OCH₃, —CH₃, and —CF₃.

In some embodiments, R⁴ is H, F, Cl, Br, —CH₃, —OCH₃, —CF₃, or —OCF₃; R⁵ is halogen, —CH₃, —CF₃, —OCF₃, or —OCH₃.

In some embodiments, R²⁰ is —CH₃, —CH₂CH₃, cyclopropyl, cyclobutyl, cyclopentyl, cyclohexyl, —CH₂OCH₃, —CH₂O-(substituted or unsubstituted phenyl), —CH(CH₃)—O-(substituted or unsubstituted phenyl), —C(CH₃)₂—O-(substituted or unsubstituted phenyl), —CH₂OCH₂-(substituted or unsubstituted phenyl), wherein if the phenyl of R²⁰ is substituted, then the phenyl is substituted with 0, 1, or 2 R²¹ groups.

In some embodiments, R²⁰ is —CH₃, cyclopropyl, —CH₂OCH₃, —CH₂O-(substituted or unsubstituted phenyl), —CH₂OCH₂-(substituted or unsubstituted phenyl), wherein if the phenyl of R²⁰ is substituted, then the phenyl is substituted with 1, or 2 R²¹ groups. In some embodiments, R²⁰ is cyclopropyl.

In some embodiments, R²⁰ is —OC₁-C₄alkyl, —O—CH₂-(substituted or unsubstituted phenyl), or —O—CH(CH₃)-(substituted or unsubstituted phenyl); wherein if the phenyl of R²⁰ is substituted, then the phenyl is substituted with 1 or 2 R²¹ groups.

In some embodiments, R²⁰ is —O—CH₂-(substituted or unsubstituted phenyl), wherein if the phenyl of R²⁰ is substituted, then the phenyl is substituted with 1 or 2 R²¹ groups.

In some embodiments, R²⁰ is —NR¹⁶C₁-C₄alkyl, —NR¹⁶—CH₂-(substituted or unsubstituted phenyl), or —NR¹⁶—CH(CH₃)-(substituted or unsubstituted phenyl), wherein if the phenyl of R²⁰ is substituted then the phenyl is substituted with 1, or 2 R²¹ groups; R¹⁶ is H, —CH₃, or —CH₂CH₃.

In some embodiments, R²⁰ is —NH—CH₂-(substituted or unsubstituted phenyl), wherein if the phenyl of R²⁰ is substituted, then the phenyl is substituted with 1, or 2 R²¹ groups. In some embodiments, R²⁰ is —NH—CH₂-phenyl.

In some embodiments, R⁴ is F, Cl, Br, —CH₃, —OCH₃, —CF₃, or —OCF₃.

In some embodiments, R⁵ is F, Cl, Br, —CH₃, —CF₃, —OCF₃, or —OCH₃.

In some embodiments, R¹¹ is —CH₃, —CH₂CH₃, or —CH₂CF₃.

In some embodiments, each R²¹ is independently selected from F, Cl, Br, —OH, —OCH₃, —CH₃, and —CF₃. In some embodiments, each R²¹ is independently selected from F, Cl, Br, —OH, —OCH₃, —OCH₂CH₃, —CH₃, and —CF₃. In some embodiments, each R²¹ is independently selected from F, Cl, and Br.

In some embodiments, R⁴ is —OCH₃. In some embodiments, R⁵ is —CF₃.

In some embodiments, R¹¹ is —CH₃, or —CH₂CH₃. In some embodiments, R¹¹ is —CH₂CH₃.

In some embodiments, R⁴ is H, F, Cl, Br, —OH, —CH₃, —OCH₃, —CF₃, or —OCF₃;

In some embodiments, R¹¹ is cyclopropyl, cyclobutyl, or cyclopentyl.

In some embodiments, R⁵ is cyclopropyl, cyclobutyl, cyclopentyl, cyclohexyl, pyrrolidinyl, piperidinyl, morpholinyl, piperazinyl, thiomorpholinyl, or a substituted or unsubstituted group selected from phenyl, naphthyl, furanyl, thienyl, pyrrolyl, oxazolyl, thiazolyl, imidazolyl, pyrazolyl, isoxazolyl, isothiazolyl, oxadiazolyl, triazolyl, tetrazolyl, pyridinyl, pyridazinyl, pyrazinyl, pyrimidinyl, triazinyl, indolyl, benzofuranyl, benzothienyl, indazolyl, benzimidaolyl, benzthiazolyl, quinolinyl, isoquinolinyl, cinnolinyl, phthalazinyl, quinazolinyl, and quinoxalinyl, where if R⁵ is substituted, then R⁵ is substituted with 1, or 2 R²¹ groups.

In some embodiments, R⁵ is a substituted or unsubstituted group selected from phenyl, naphthyl, pyrrolyl, oxazolyl, thiazolyl, imidazolyl, pyrazolyl, isoxazolyl, isothiazolyl, oxadiazolyl, triazolyl, tetrazolyl, pyridinyl, pyridazinyl, pyrazinyl, pyrimidinyl, triazinyl, indolyl, benzofuranyl, benzothienyl, indazolyl, benzimidaolyl, benzthiazolyl, quinolinyl, isoquinolinyl, cinnolinyl, phthalazinyl, quinazolinyl, and quinoxalinyl, where if R⁵ is substituted, then R⁵ is substituted with 1 or 2 R²¹ groups.

In some embodiments, R⁵ is a substituted or unsubstituted group selected from pyrrolyl, oxazolyl, thiazolyl, imidazolyl, pyrazolyl, isoxazolyl, isothiazolyl, oxadiazolyl, triazolyl, tetrazolyl, pyridinyl, pyridazinyl, pyrazinyl, pyrimidinyl, triazinyl, indolyl, benzofuranyl, benzothienyl, indazolyl, benzimidaolyl, benzthiazolyl, quinolinyl, isoquinolinyl, where if R⁵ is substituted, then R⁵ is substituted with 1 or 2 R²¹ groups.

In some embodiments, R⁵ is a substituted or unsubstituted pyridinyl, where if R⁵ is substituted, then R⁵ is substituted with 1 or 2 R²¹ groups.

In some embodiments, R⁴ is F, Cl, Br, —CH₃, —OCH₃, —CF₃, or —OCF₃; R^{H} is —CH₃, —CH₂CH₃, or —CH₂CF₃;

In some embodiments, R⁴ is —OCH₃; R¹¹ is —CH₃, or —CH₂CH₃.

In some embodiments, R⁵ is a substituted or unsubstituted group selected from pyrrolyl, oxazolyl, thiazolyl, imidazolyl, pyrazolyl, isoxazolyl, isothiazolyl, pyridinyl, pyridazinyl, pyrazinyl, pyrimidinyl, quinolinyl, and isoquinolinyl, where if R⁵ is substituted, then R⁵ is substituted with 1, or 2 R²¹ groups.

In some embodiments, R⁵ is cyclopropyl, phenyl, pyrrolidin-1-yl, pyrazol-1-yl, 1H-pyrazol-4-yl, 1-methyl-1H-pyrazol-4-yl, oxazol-2-yl, pyridin-2-yl, 6-ethoxy-pyridin-3-yl, 5-fluoro-pyridin-2-yl, 5-methoxy-pyrimidin-2-yl, or quinolin-7-yl.

In some embodiments, R⁵ is pyrazol-1-yl, 1H-pyrazol-4-yl, 1-methyl-1H-pyrazol-4-yl, oxazol-2-yl, pyridin-2-yl, 6-ethoxy-pyridin-3-yl, 5-fluoro-pyridin-2-yl, 5-methoxy-pyrimidin-2-yl, or quinolin-7-yl.

In some embodiments, R⁵ is substituted or unsubstituted pyridin-2-yl, substituted or unsubstituted pyridin-3-yl, or substituted or unsubstituted pyridin-4-yl, where if R⁵ is substituted, then R⁵ is substituted with 1 or 2 R²¹ groups. In some embodiments, R⁵ is pyridin-2-yl, 6-ethoxy-pyridin-3-yl, 5-fluoro-pyridin-2-yl, or 5-methoxy-pyrimidin-2-yl. In some embodiments, R⁵ is pyridin-2-yl, 6-methyl-pyridin-3-yl, 6-ethyl-pyridin-3-yl, 6-methoxy-pyridin-3-yl, 6-ethoxy-pyridin-3-yl, 5-fluoro-pyridin-2-yl, 5-methyl-pyridin-2-yl, 5-ethyl-pyridin-2-yl, 5-methoxy-pyrimidin-2-yl or 5-ethoxy-pyrimidin-2-yl. In some embodiments, R⁵ is 6-ethoxy-pyridin-3-yl.

In some embodiments, the DP₂ receptor antagonist is [2′-(3-benzyl-1-ethyl-ureidomethyl)-6-methoxy-4′-trifluoromethyl-biphenyl-3-yl]-acetic acid, {2′-[(N-cyclopropanecarbonyl-N-ethyl-amino)-methyl]-6-methoxy-4′-trifluoromethyl-biphenyl-3-yl}-acetic acid, [2′-[(N-cyclopropanecarbonyl-N-ethyl-amino)-methyl]-4′-(6-ethoxy-pyridin-3-yl)-6-methoxy-biphenyl-3-yl]-acetic acid, or a pharmaceutically acceptable salt, pharmaceutically acceptable solvate, or prodrug thereof.

In some embodiments, the DP₂ receptor antagonist is a compound having the structure of Formula (II), pharmaceutically acceptable salt, pharmaceutically acceptable solvate, or prodrug thereof:

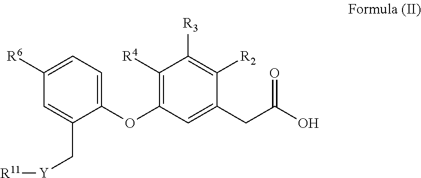

wherein,
each of R², R³, and R⁴ is independently H, F, Cl, Br, I, —CN, —OR¹², —C₁-C₆alkyl, C₁-C₆fluoroalkyl, C₁-C₆fluoroalkoxy, C₁-C₆alkoxy, or C₁-C₆heteroalkyl;
R⁶ is F, Cl, Br, I, —CN, —NO₂, —OH, —O(C₁-C₆alkyl), —S(═O)₂R¹², —N(C₁-C₄alkyl)S(═O)₂R¹², —NHS(═O)₂R¹², —S(═O)₂N(R¹³)₂, —C(═O)R¹², —CO₂(C₁-C₆alkyl), —NH₂, —C(═O)NH(R¹³), —C(═O)N(R¹³)₂, —OC(═O)NH(R¹³), —OC(═O)N(R¹³)₂, —N(C₁-C₄alkyl)C(═O)N(R¹³)₂, —NHC(═O)N(R¹³)₂, —NHC(═O)NH(R¹³), —N(C₁-C₄alkyl)C(═O)R¹², —NHC(═O)R¹², —NH—C₁-C₄alkyl-C(═O)R¹², —N(C₁-C₄alkyl)C(═O)OR¹², —NHC(═O)OR¹², C₁-C₆alkyl, C₁-C₆fluoroalkyl, C₁-C₆fluoroalkoxy, C₁-C₆alkoxy, C₁-C₆heteroalkyl, a substituted or unsubstituted C₂-C₁₀heterocycloalkyl, a substituted or unsubstituted phenyl, or a substituted or unsubstituted monocyclic or bicyclic heteroaryl containing 0-3 heteroatoms selected from N, O or S;
R¹¹ is C₁-C₆alkyl, C₁-C₆haloalkyl, C₁-C₆heteroalkyl, a substituted or unsubstituted phenyl, a substituted or unsubstituted monocyclic or bicyclic heteroaryl containing 0-3 heteroatoms selected from N, O or S, a substituted or unsubstituted —C₁-C₄alkyl-phenyl, —C₁-C₆alkylene-N(R¹⁷)₂, —C₁-C₆alkylene-C(═O)O—R¹⁷, or —C₁-C₆alkylene-C(═O)N(R¹⁷)₂; R¹⁷ is H, or C₁-C₆alkyl;
R¹² is C₁-C₆alkyl, C₁-C₆heteroalkyl, C₁-C₆fluoroalkyl, a substituted or unsubstituted C₃-C₁₀cycloalkyl, a substituted or unsubstituted C₂-C₁₀heterocycloalkyl, a substituted or unsubstituted phenyl, a substituted or unsubstituted benzyl, a substituted or unsubstituted monocyclic heteroaryl containing 0-3 heteroatoms selected from N, O or S, a substituted or unsubstituted —C₁-C₄alkyl-C₃-C₁₀cycloalkyl, a substituted or unsubstituted —C₁-C₄alkyl-phenyl, or a substituted or unsubstituted —C₁-C₄alkyl-(monocyclic heteroaryl containing 0 to 3 heteroatoms selected from N, O or S);
each R¹³ is independently selected from H, C₁-C₆alkyl, C₁-C₆heteroalkyl, C₁-C₆fluoroalkyl, a substituted or unsubstituted C₃-C₁₀cycloalkyl, a substituted or unsubstituted C₂-C₁₀heterocycloalkyl, a substituted or unsubstituted phenyl, a substituted or unsubstituted benzyl, a substituted or unsubstituted monocyclic heteroaryl containing 0-3 heteroatoms selected from N, O or S, a substituted or unsubstituted —C₁-C₄alkyl-C₃-C₁₀cycloalkyl, a substituted or unsubstituted —C₁-C₄alkyl-C₂-C₁₀heterocycloalkyl, a substituted or unsubstituted —C₁-C₄alkyl-phenyl, and a substituted or unsubstituted —C₁-C₄alkyl-(monocyclic heteroaryl containing 0 to 3 heteroatoms selected from N, O or S); or
two R¹³ groups attached to the same N atom are taken together with the N atom to which they are attached to form a substituted or unsubstituted C₂-C₁₀heterocycloalkyl;
Y is —S—, —S(═O)—, or —S(═O)₂—.

In some embodiments, R⁶ is F, Cl, Br, I, —CN, —NO₂, —S(═O)₂R¹², —N(C₁-C₄alkyl)S(═O)₂R¹², —NHS(═O)₂R¹², —S(═O)₂N(R¹³)₂, —C(═O)R¹², —CO₂(C₁-C₆alkyl), —NH₂, —C(═O)NH(R¹³), —C(═O)N(R¹³)₂, —N(C₁-C₄alkyl)C(═O)N(R¹³)₂, —NHC(═O)N(R¹³)₂, —NHC(═O)NH(R¹³), —N(C₁-C₄alkyl)C(═O)R¹², —NHC(═O)R¹², —NH—C₁-C₄alkyl-C(═O)R¹², —N(C₁-C₄alkyl)C(═O)OR¹², —NHC(═O)OR¹², C₁-C₆alkyl, C₁-C₆fluoroalkyl, C₁-C₆fluoroalkoxy, C₁-C₆alkoxy, C₁-C₆heteroalkyl, a substituted or unsubstituted C₂-C₁₀heterocycloalkyl, a substituted or unsubstituted phenyl, or a substituted or unsubstituted monocyclic heteroaryl containing 0-3 heteroatoms selected from N, O or S.

In some embodiments, R¹¹ is isopropyl, tert-butyl, —CH₂CF₃, —CH₂CO₂H, —CH₂CH₂N(CH₃)₂, phenyl, 4-chlorophenyl, benzyl, phenethyl, thiazol-2-yl, 5-methyl-[1,3,4]thiadiazol-2-yl, pyridin-2-yl, or quinolin-2-yl.

In some embodiments, R⁶ is —NO₂, —N(C₁-C₄alkyl)S(═O)₂R¹², —NHS(═O)₂R¹², —N(R¹³)₂, —N(C₁-C₄alkyl)C(═O)N(R¹³)₂, —NHC(═O)N(R¹³)₂, —N(C₁-C₄alkyl)C(═O)R¹², —NHC(═O)R¹², —NH—C₁-C₄alkyl-C(═O)R¹², —N(C₁-C₄alkyl)C(═O)OR¹², or —NHC(═O)OR¹².

In some embodiments, R⁶ is —N(C₁-C₄alkyl)C(═O)R¹² or —NHC(═O)R¹².

In some embodiments, each of R², R³, and R⁴ is independently H, F, Cl, Br, I, —CN, —OCH₃, —CH₃, —CH₂CH₃, —CHCH₂, —CHF₂, —CF₃, —OCHF₂, or —OCF₃.

In some embodiments, R² is H. In some embodiments, R³ is H.

In some embodiments, R⁴ is H, halogen, —CN, —OH, C₁-C₄alkyl, C₁-C₄fluoroalkyl, C₁-C₄fluoroalkoxy, C₁-C₄alkoxy, or C₁-C₄heteroalkyl; R⁶ is —NR¹³S(═O)₂R¹², —S(═O)₂N(R¹²)(R¹³), —N(R¹²)(R¹³), —C(═O)N(R¹²)(R¹³), —NHC(═O)N(R¹²)(R¹³), —NR¹³C(═O)R¹², or —NR¹³C(═O)OR¹²; R¹¹ is C₁-C₆alkyl, C₁-C₆haloalkyl, C₁-C₆heteroalkyl, C₃-C₆cycloalkyl, a substituted or unsubstituted phenyl, a substituted or unsubstituted naphthyl, a substituted or unsubstituted 5-membered heteroaryl, a substituted or unsubstituted 6-membered heteroaryl, or —C₁-C₄alkyl-(substituted or unsubstituted phenyl); R¹² is C₁-C₆alkyl, C₁-C₆heteroalkyl, C₁-C₆fluoroalkyl, C₃-C₆cycloalkyl, a substituted or unsubstituted phenyl, a substituted or unsubstituted naphthyl, a substituted or unsubstituted benzyl, a substituted or unsubstituted 6-membered heteroaryl, or —C₁-C₄alkyl-(substituted or unsubstituted phenyl); R¹³ is H or C₁-C₄alkyl; or R¹² and R¹³ attached to the same N atom are taken together with the N atom to which they are attached to form a substituted or unsubstituted C₂-C₆heterocycloalkyl.

In some embodiments, R⁴ is H, F, Cl, Br, —OH, C₁-C₄alkyl, C₁-C₄fluoroalkyl, C₁-C₄fluoroalkoxy, or C₁-C₄alkoxy.

In some embodiments, R¹¹ is C₁-C₆alkyl, C₁-C₆haloalkyl, C₃-C₆cycloalkyl, a substituted or unsubstituted phenyl, or —C₁-C₄alkyl-(substituted or unsubstituted phenyl).

In some embodiments, R⁴ is H, F, Cl, Br, —OCH₃, —CH₃, —CH₂CH₃, —CHCH₂, —CHF₂, —CF₃, —OCHF₂, or —OCF₃.

In some embodiments, R¹² is C₁-C₆alkyl, C₁-C₆heteroalkyl, C₁-C₆fluoroalkyl, C₃-C₆cycloalkyl, a substituted or unsubstituted phenyl, a substituted or unsubstituted benzyl, or —C₁-C₄alkyl-(substituted or unsubstituted phenyl); R¹³ is H or —CH₃.

In some embodiments, R⁶ is —NR¹³S(═O)₂R¹², —N(R¹²)(R¹³), —C(═O)N(R¹²)(R¹³), —NHC(═O)N(R¹²)(R¹³), —NR¹³C(═O)R¹², or —NR¹³C(═O)OR¹².

In some embodiments, R¹¹ is C₁-C₆alkyl, C₁-C₆haloalkyl, a substituted or unsubstituted phenyl, or —C₁-C₄alkyl-(substituted or unsubstituted phenyl).

In some embodiments, R⁴ is F, Cl, Br, —OCH₃, —CH₃, —CH₂CH₃, —CHCH₂, —CHF₂, —CF₃, —OCHF₂, or —OCF₃. In some embodiments, R⁴ is —OCH₃.

In some embodiments, R⁶ is —NR¹³C(═O)R¹².

In some embodiments, R¹² is C₁-C₆alkyl, C₃-C₆cycloalkyl, a substituted or unsubstituted phenyl, or a substituted or unsubstituted benzyl.

In some embodiments, R¹¹ is —CH₂CH₃, —CH(CH₃)₂, —C(CH₃)₃, —CH₂CF₃, a substituted or unsubstituted phenyl, —C₁-C₂alkyl-(substituted or unsubstituted phenyl).

In some embodiments, R¹² is —CH(CH₃)₃, —C(CH₃)₃, —CH₂CH(CH₃)₂, —CH₂C(CH₃)₃, cyclopropyl, cyclobutyl, cyclopentyl, cyclohexyl, a substituted or unsubstituted phenyl, or a substituted or unsubstituted benzyl.

In some embodiments, R¹¹ is —CH₂CH₃, —CH(CH₃)₂, —C(CH₃)₃, or —CH₂CF₃; R¹² is —CH(CH₃)₂, —C(CH₃)₃, —CH₂CH(CH₃)₂, —CH₂C(CH₃)₃, or a substituted or unsubstituted phenyl; R¹³ is H.

In some embodiments, R⁴ is F, Cl, —OCH₃, —CF₃, or —OCF₃; R¹¹ is —C(CH₃)₃; R¹² is —C(CH₃)₃; R¹³ is H.

In some embodiments, the DP₂ receptor antagonist is {3-[2-tert-butylsulfanylmethyl-4-(2,2-dimethyl-propionylamino)-phenoxy]-4-methoxy-phenyl}-acetic acid, or a pharmaceutically acceptable salt, pharmaceutically acceptable solvate, or prodrug thereof.

In some embodiments, the DP₂ receptor antagonist is a compound having the structure of Formula (III), pharmaceutically acceptable salt, pharmaceutically acceptable solvate, N-oxide, or prodrug thereof:

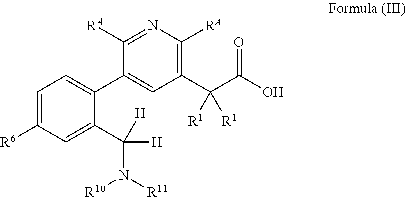

wherein,
each R¹ is independently selected from H and —CH₃;
each R^{A} is independently selected from H, halogen, —CN, —OH, —OR¹², —N(R¹³)₂, C₁-C₆alkyl, C₁-C₆fluoroalkyl, C₁-C₆fluoroalkoxy, C₁-C₆alkoxy, and C₁-C₆heteroalkyl.
R⁶ is selected from halogen, —CN, —NO₂, —OH, —OR¹², —SR¹², —S(═O)R¹², —S(═O)₂R¹², —NHS(═O)₂R¹², —N(C₁-C₆alkyl)S(═O)₂R¹², —S(═O)₂N(R¹³)₂, —C(═O)R¹², —OC(═O)R¹², —CO₂R¹³, —OCO₂R¹³, —N(R¹³)₂, —C(═O)N(R¹³)₂, —OC(═O)N(R¹³)₂, —NHC(═O)N(R¹³)₂, —N(C₁-C₆alkyl)C(═O)N(R¹³)₂, —NHC(═O)R¹², —N(C₁-C₆alkyl)C(═O)R¹², —NHC₁-C₄alkyl-C(═O)R¹², —C₁-C₄alkyl-N(R¹³)₂, —C₁-C₄alkyl—NHC(═O)R¹², —C₁-C₄alkyl—NHS(═O)₂R¹², —NHC(═O)OR¹², —N(C₁-C₆alkyl)C(═O)OR¹², C₁-C₆alkyl, C₁-C₆fluoroalkyl, C₁-C₆fluoroalkoxy, C₁-C₆alkoxy, C₁-C₆heteroalkyl, a substituted or unsubstituted cycloalkyl, and a substituted or unsubstituted heterocycloalkyl;
R¹⁰ is —C(═O)R¹⁴, —C(═O)OR¹⁵, or —C(═O)N(R¹⁶)₂;
   R¹⁴ is C₁-C₆alkyl, C₁-C₆fluoroalkyl, C₁-C₆heteroalkyl, or a C₃-C₆cycloalkyl; or
   R¹⁴ is a substituted or unsubstituted aryl, a substituted or unsubstituted heteroaryl, a substituted or unsubstituted —C₁-C₄alkyl-aryl or a substituted or unsubstituted —C₁-C₄alkyl-heteroaryl;
   R¹⁵ is C₁-C₆alkyl, C₁-C₆fluoroalkyl, C₁-C₆heteroalkyl, or a C₃-C₆cycloalkyl; or
   R¹⁵ is a substituted or unsubstituted aryl, a substituted or unsubstituted heteroaryl, a substituted or unsubstituted —C₁-C₄alkyl-aryl, or a substituted or unsubstituted —C₁-C₄alkyl-heteroaryl;
   each R¹⁶ is independently H, —CN, C₁-C₆alkyl, C₁-C₆fluoroalkyl, C₁-C₆heteroalkyl, or a C₃-C₆cycloalkyl; or two R¹⁶ groups attached to the same N atom are taken together with the N atom to which they are attached to form an optionally substituted heterocycloalkyl; or each R¹⁶ is independently H, C₁-C₆alkyl, C₁-C₆fluoroalkyl, a substituted or unsubstituted aryl, a substituted or unsubstituted heteroaryl, a substituted or unsubstituted —C₁-C₄alkyl-aryl, or a substituted or unsubstituted —C₁-C₄alkyl-heteroaryl;
   R″ is a substituted or unsubstituted aryl, a substituted or unsubstituted heteroaryl, a substituted or unsubstituted —C₁-C₄alkyl-aryl, or a substituted or unsubstituted —C₁-C₄alkyl-heteroaryl; or
   R¹¹ is C₁-C₆alkyl, C₁-C₆haloalkyl, or C₁-C₆heteroalkyl.

In some embodiments, each R^{A} is H, halogen, —CN, —OH, C₁-C₄alkyl, C₁-C₄fluoroalkyl, C₁-C₄fluoroalkoxy, or C₁-C₄alkoxy; R⁶ is H, halogen, —CN, tetrazolyl, —OH, —SR¹³, —S(═O)R¹², —S(═O)₂R¹², —NHS(═O)₂R¹², —C(═O)R¹², —OC(═O)R¹², —CO₂R¹³, —N(R¹³)₂, —C(═O)N(R¹³)₂, —NHC(═O)R¹², C₁-C₄alkyl, C₁-C₄fluoroalkyl, C₁-C₄fluoroalkoxy, C₁-C₄alkoxy, or C₁-C₄heteroalkyl; R¹⁰ is —C(═O)C₁-C₄alkyl, —C(═O)C₁-C₄fluoroalkyl, —C(═O)C₃-C₆cycloalkyl, —C(═O)(a substituted or unsubstituted phenyl), —C(═O)(a substituted or unsubstituted 6-membered heteroaryl containing 1 or 2 N atom), —C(═O)C₁-C₂alkyl-(substituted or unsubstituted phenyl), —C(═O)—C₁-C₂alkyl-(substituted or unsubstituted 6-membered heteroaryl containing 1 or 2 N atom), —C(═O)C₁-C₂alkyl-O—C₁-C₄alkyl, —C(═O)—O—C₁-C₄alkyl, —C(═O)—O—C₁-C₂alkyl-(substituted or unsubstituted phenyl), —C(═O)—NR¹⁶C₁-C₄alkyl, or —C(═O)—NR¹⁶C₁-C₂alkyl-(substituted or unsubstituted phenyl); R¹¹ is C₁-C₄alkyl, C₁-C₄haloalkyl, C₃-C₆cycloalkyl, or substituted or unsubstituted benzyl; R¹² is C₁-C₄alkyl, C₁-C₄heteroalkyl, or C₁-C₄fluoroalkyl; each R¹³ is independently selected from H, C₁-C₄alkyl, C₁-C₄heteroalkyl, C₁-C₄fluoroalkyl, or substituted or unsubstituted benzyl; R¹⁶ is H or C₁-C₄alkyl; where each substituted phenyl or substituted heteroaryl is substituted with 1 or 2 R^{C}, where each R^{C} is independently selected from halogen, —OH, C₁-C₄alkyl, C₁-C₄fluoroalkoxy, C₁-C₄fluoroalkoxy, and C₁-C₄alkoxy.

In some embodiments, R¹⁰ is —C(═O)C₁-C₄alkyl, —C(═O)C₃-C₆cycloalkyl, —C(═O)C₁-C₂alkyl-(substituted or unsubstituted phenyl), —C(═O)—C₁-C₂alkyl-(substituted or unsubstituted 6-membered heteroaryl containing 1 or 2 N atom), —C(═O)—O—C₁-C₂alkyl-(substituted or unsubstituted phenyl), or —C(═O)—NR¹⁶C₁-C₂alkyl-(substituted or unsubstituted phenyl).

In some embodiments, R¹⁰ is —C(═O)C₁-C₄alkyl, —C(═O)C₃-C₆cycloalkyl, —C(═O)CH₂-(substituted or unsubstituted phenyl), —C(═O)—CH₂-(substituted or unsubstituted 6-membered heteroaryl containing 1 or 2 N atom), —C(═O)—O—CH₂-(substituted or unsubstituted phenyl), or —C(═O)—NHCH₂-(substituted or unsubstituted phenyl). In some embodiments, R¹⁰ is —C(═O)C₁-C₄alkyl, —C(═O)C₃-C₆cycloalkyl, —C(═O)CH₂-(substituted or unsubstituted phenyl), —C(═O)—O—CH₂-(substituted or unsubstituted phenyl), or —C(═O)—NHCH₂-(substituted or unsubstituted phenyl). In some embodiments, R¹⁰ is —C(═O)—O—CH₂-(substituted or unsubstituted phenyl). In some embodiments, R¹⁰ is —C(═O)CH₃, —C(═O)CH₂CH₃, —C(═O)cyclopropyl, —C(═O)CH₂OCH₃, or —C(═O)CH₂OCH₂CH₃.

In some embodiments, each R^{A} is independently selected from H, F, Cl, Br, I, —CN, —OH, —OCH₃, —OCH₂CH₃, —CH₃, —CH₂CH₃, —CF₃, —CHF₂, —CH₂F, and —OCF₃.

In some embodiments, R⁶ is H, —CF₃, —CO₂H, Br, —NH—C(═O)—CH₃, —NH—C(═O)—OCH₃, —NH—SO₂CH₃, —SCH₃, —SO₂CH₃, —NH—(C═O)—CH₃, —NH—SO₂—CH₃, or —C(CH₃)₂—(OH). In some embodiments, R⁶ is F, Cl, —CH₃, —CF₃, —OCF₃, or —OCH₃. In some embodiments, R⁶ is —CF₃.

In some embodiments, R¹¹ is C₁-C₆alkyl. In some embodiments, R¹¹ is —CH₂CH₃.

In some embodiments, the DP₂ receptor antagonist is (5-{2-[(N-benzyloxycarbonyl-N-ethyl-amino)-methyl]-4-trifluoromethyl-phenyl}-pyridin-3-yl)-acetic acid, or a pharmaceutically acceptable salt, pharmaceutically acceptable solvate, or prodrug thereof.

In some embodiments, the DP₂ receptor antagonist is a compound having the structure of Formula (IV), pharmaceutically acceptable solvate, pharmaceutically acceptable salt, N-oxide, or prodrug thereof:

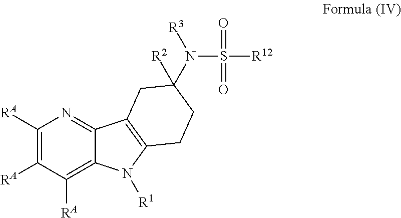

wherein,
R¹ is L¹—X¹; L¹ is C₁-C₆alkyl; X¹ is CO₂H, or —CO₂(C₁-C₆alkyl); each R^{A} is independently selected from H, halogen, —CN, —OH, C₁-C₆alkyl, C₁-C₆fluoroalkyl, C₁-C₆fluoroalkoxy, and C₁-C₆alkoxy;
R² is H or —CH₃;
R³ is H or C₁-C₆alkyl;
R² is C₁-C₆alkyl, C₁-C₆fluoroalkyl, C₁-C₆heteroalkyl, an optionally substituted C₃-C₁₀cycloalkyl, an optionally substituted aryl, an optionally substituted heteroaryl, an optionally substituted —C₁-C₆alkyl-cycloalkyl, an optionally substituted —C₁-C₆alkyl-phenyl, or an optionally substituted —C₁-C₆alkyl-heteroaryl.

In some embodiments, R¹² is C₁-C₆alkyl, an optionally substituted phenyl, an optionally substituted naphthyl, or an optionally substituted heteroaryl containing 0-3 N atoms.

In some embodiments, R¹² is an optionally substituted phenyl, an optionally substituted naphthyl, an optionally substituted monocyclic heteroaryl containing 0-3 N atoms or an optionally substituted bicyclic heteroaryl containing 0-3 N atoms.

In some embodiments, each R^{A} is independently selected from H, F, Cl, Br, I, —CN, —OH, —OCH₃, —CH₃, and —CF₃. In some embodiments, each R^{A} is H.

In some embodiments, L¹ is —CH₂—, —CH(CH₃)—, —C(CH₃)₂—, or —CH₂CH₂—. In some embodiments, L¹ is —CH₂— or —CH₂CH₂—. In some embodiments, L¹ is —CH₂—. In some embodiments, L¹ is —CH₂CH₂—.

In some embodiments, R¹² is a substituted or unsubstituted phenyl, where if R¹² is substituted then R¹² is substituted with 1 or 2 groups selected from F, Cl, Br, I, —CN, _{—NH}₂, —OH, —NH(CH₃), —N(CH₃)₂, —CH₃, —CF₃, —OCH₃, and —OCF₃. In some embodiments, R¹² is a substituted or unsubstituted phenyl, where if R¹² is substituted then R¹² is substituted with 1 group selected from F, Cl, Br, I, —CN, —NH₂, —OH, —NH(CH₃), —N(CH₃)₂, —CH₃, —CF₃, —OCH₃, and —OCF₃. In some embodiments, R¹² is 4-fluorophenyl.

In some embodiments, the DP₂ receptor antagonist is {8-[(4-fluoro-benzenesulfonyl)-methyl-amino]-6,7,8,9-tetrahydro-pyrido[3,2b]indol-5-yl}-acetic acid, (R)-{8-[(4-fluoro-benzenesulfonyl)-methyl-amino]-6,7,8,9-tetrahydro-pyrido[3,2-b]indol-5-yl}-acetic acid, (S)-{8-[(4-fluoro-benzenesulfonyl)-methyl-amino]-6,7,8,9-tetrahydro-pyrido[3,2-b]indol-5-yl}-acetic acid, or a pharmaceutically acceptable salt, pharmaceutically acceptable solvate, or prodrug thereof. In some embodiments, the DP₂ receptor antagonist is {8-[(4-fluoro-benzenesulfonyl)-methyl-amino]-6,7,8,9-tetrahydro-pyrido[3,2-b]indol-5-yl}-acetic acid, or a pharmaceutically acceptable salt, pharmaceutically acceptable solvate, or prodrug thereof. In some embodiments, the DP₂ receptor antagonist is (R)-{8-[(4-fluoro-benzenesulfonyl)-methyl-amino]-6,7,8,9-tetrahydro-pyrido[3,2-b]indol-5-yl}-acetic acid, or a pharmaceutically acceptable salt, pharmaceutically acceptable solvate, or prodrug thereof. In some embodiments, the DP₂ receptor antagonist is (S)-{8-[(4-fluoro-benzenesulfonyl)-methyl-amino]-6,7,8,9-tetrahydro-pyrido[3,2-b]indol-5-yl}-acetic acid, or a pharmaceutically acceptable salt, pharmaceutically acceptable solvate, or prodrug thereof.

Any combination of the groups described above for the various variables is contemplated herein. Throughout the specification, groups and substituents thereof are chosen by one skilled in the field to provide stable moieties and compounds.

In some embodiments, the DP₂ receptor antagonist compounds are included in the formulations described herein as pharmaceutically acceptable salts, and/or pharmaceutically acceptable solvates. In some embodiments, the DP₂ receptor antagonist compounds are included in the formulations described herein as pharmaceutically acceptable salts. In some embodiments, DP₂ receptor antagonist compounds are included in the formulations described herein in free acid form or free base form.

In some embodiments, the DP₂ receptor antagonist compounds described herein possess one or more stereocenters and each center exists independently in either the R or S configuration. The compounds presented herein include all diastereomeric, enantiomeric, and epimeric forms as well as the appropriate mixtures thereof.

### Combination Therapy

In one aspect, pharmaceutical compositions and methods disclosed herein include an additional therapeutic agent. In one aspect, the additional therapeutic agent is a therapeutic agent other than a DP₂ receptor antagonist compound.

In one aspect, the dermal formulations disclosed herein that include a DP₂ receptor antagonist compound are co-administered with (either separately or in the same formulation) a therapeutic agent selected from: antibiotics (e.g., polymyxin B sulfate/bacitracin zinc, polymyxin B/neomycin/gramicidin, polymyxin B/trimethoprim, polymyxin B/bacitracin, fluoroquinolones (e.g., ciprofloxacin, moxifloxacin, ofloxacin, gatifloxacin, levofloxacin), aminoglycosides (e.g. tobramycin, azithromycin, gentamicin, erythromycin, bacitracin); anti-Fungal Agents (e.g., amphotericin B, intraconazole, fluconazole, voriconazole); steroid anti-inflammatory agents (e.g., fluorometholone acetate, prednisolone acetate, loteprednol etabonate, prednisolone sodium phosphate, prednisolone sodium, rimexolone, fluorometholone acetate); non-steroidal anti-inflammatory agents (e.g., nepafenac, ketorolac tromethamine, bromfenac, diclofenac sodium, ketorolac tromethamine, ketotifen fumarate); antihistamines (e.g., emedastine difumarate, olopatadine hydrochloride, epinastine HCl, azelastine hydrochloride, ketotifen fumarate); antivirals (e.g., acyclovir, vidarabine, trifluridine); mast cell stabilizers (e.g., lodoxamide tromethamine, nedocromil sodium, cromolyn sodium, pemirolast potassium), cyclosporine, and leukotriene modulators (e.g. 5-LO inhibitiors, FLAP inhibitor compounds, LTA₄ hydrolase inhibitors, leukotriene receptor antagonist (e.g. CysLT₁ receptor antagonists, BLT₁R antagonists)).

In one aspect, the dermal formulations disclosed herein that include a DP₂ receptor antagonist compound are co-administered with (either separately or in the same formulation) an antibiotic. Antibiotics include, but are not limited to, polymyxin B sulfate/bacitracin zinc, polymyxin B/neomycin/gramicidin, polymyxin B/trimethoprim, polymyxin B/bacitracin, fluoroquinolones (e.g., ciprofloxacin, moxifloxacin, ofloxacin, gatifloxacin, levofloxacin), aminoglycosides (e.g. tobramycin, azithromycin, gentamicin, erythromycin, bacitracin.

In one aspect, the dermal formulations disclosed herein that include a DP₂ receptor antagonist compound are co-administered with (either separately or in the same formulation) an anti-fungal agent. Anti-fungal agents include, but are not limited to, amphotericin B, intraconazole, fluconazole, voriconazole.

In one aspect, the dermal formulations disclosed herein that include a DP₂ receptor antagonist compound are co-administered with (either separately or in the same formulation) a steroid anti-inflammatory agent. Steroid anti-inflammatory agents include, but are not limited to, betamethasone, prednisone, alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

In one aspect, the dermal formulations disclosed herein that include a DP₂ receptor antagonist compound are co-administered with (either separately or in the same formulation) a non-steroidal anti-inflammatory agent (NSAID). NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, lumiracoxib, CS-502, JTE-522, L-745,337 and NS398).

In one aspect, the dermal formulations disclosed herein that include a DP₂ receptor antagonist compound are co-administered with (either separately or in the same formulation) an antihistamine. Antihistamines include, but are not limited to, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine, and triprolidine.

In one aspect, the dermal formulations disclosed herein that include a DP₂ receptor antagonist compound are co-administered with (either separately or in the same formulation) an antiviral agent. Antiviral agents include, but are not limited to, acyclovir, vidarabine, trifluridine.

In one aspect, the dermal formulations disclosed herein that include a DP₂ receptor antagonist compound are co-administered with (either separately or in the same formulation) a mast cell stabilizer. Mast cell stabilizers include, but are not limited to, lodoxamide tromethamine, nedocromil sodium, cromolyn sodium, pemirolast potassium.

In one aspect, the dermal formulations disclosed herein that include a DP2 receptor antagonist compound are co-administered with (either separately or in the same formulation) cyclosporine.

In one aspect, the dermal formulations disclosed herein that include a DP₂ receptor antagonist compound are co-administered with (either separately or in the same formulation) a leukotriene modulator. Leukotriene modulators include, but are not limited to, 5-lipoxygenase inhibitors (5-LO) inhibitiors, 5-lipoxygenase activating protein (FLAP) inhibitor compounds, LTA₄ hydrolase inhibitors, leukotriene receptor antagonist (e.g. CysLT₁ receptor antagonists, BLT₁R antagonists).

In some embodiments, the second therapeutic agent is a leukotriene receptor antagonist selected from CysLT₁/CysLT₂ dual receptor antagonists, and CysLT₁ receptor antagonists. CysLT₁ receptor antagonists include, but are not limited to, zafirlukast, montelukast, prankulast, and derivatives or analogs thereof.

In some embodiments, the second therapeutic agent is a FLAP inhibitor compound that is selected from FLAP inhibitor compounds disclosed herein or known in the art. In some embodiments, the FLAP inhibitor is selected from compounds described in U.S. patent application Ser. No. 11/538,762 (issued as U.S. Pat. No. 7,405,302); U.S. patent application Ser. No. 12/131,828; U.S. patent application Ser. No. 11/553,946 (published as 2007/0105866); U.S. patent application Ser. No. 11/925,841; U.S. patent application Ser. No. 12/089,706; U.S. patent application Ser. No. 12/089,707; U.S. patent application Ser. No. 12/092,570; U.S. patent application Ser. No. 11/744,555 (published as 2007/0219206); U.S. patent application Ser. No. 11/746,010 (published as 2007/0225285); U.S. patent application Ser. No. 11/745,387 (published as 2007/0244128); U.S. patent application Ser. No. 12/257,876; U.S. patent application No. 61/055,887; U.S. patent application No. 61/055,899; International Patent Application no. PCT/US07/86188; WO 07/047207; WO07/056021; WO07/056220; WO07/056228; International Patent Application no. PCT/US08/62310; International Patent Application no. PCT/US08/062793; International Patent Application no. PCT/US08/62580; International Patent Application no. PCT/US2008/052960; International Patent Application no. PCT/US08/81190; International Patent Application no. PCT/US08/76225; each of which is herein incorporated by reference in its entirety.

In some embodiments, the second therapeutic agent is a FLAP inhibitor is selected from: MK886 (also known as 3-[3-tert-butylsulfanyl-1-(4-chloro-benzyl)-5-isopropyl-1H-indol-2-yl]-2,2-dimethyl-propionic acid); MK591 (also known as 3-[3-tert-butylsulfanyl-1-(4-chloro-benzyl)-5-(quinolin-2-ylmethoxy)-1H-indol-2-yl]-2,2-dimethyl-propionic acid); and DG031 (also known as BAY X1005; cyclopentyl-[4-(quinolin-2-ylmethoxy)-phenyl]-acetic acid), Compound A (3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid); Compound B (3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid); Compound C (3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid); Compound D (3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid); Compound E (3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid); Compound F (3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid); Compound G (2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid); Compound H (3-[3-tert-Butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid); Compound I (3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid); Compound J (3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid), Compound K (3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid), or pharmaceutically acceptable salt or N-oxide thereof.

In another aspect, the FLAP inhibitor is selected from compounds described in U.S. Pat. Nos. 4,929,626; 4970215; 5,081,138; 5,095,031; 5,204,344; 5,126,354; 5,221,678; 5,229,516; 5,272,145; 5,283,252; 5,288,743; 5,292,769; 5,304,563; 5,399,699; 5,459,150; 5,512,581; 5,597,833; 5,668,146; 5,668,150; 5,691,351; 5,714,488; 5,783,586; 5,795,900; and 5,843,968, each of which is herein incorporated by reference for the disclosure of such FLAP inhibitors).

In one aspect, a DP₂ receptor antagonist compound described herein is used in combination with a second therapeutic agent compound for the treatment of any dermal disease or condition described herein. In some instances, the ratio of the amount of a DP₂ receptor antagonist compound to a second therapeutic agent compound in any dermal formulation described herein is from about 10:1 to about 1:10. In some instances, the ratio of the amount of a DP₂ receptor antagonist compound to a second therapeutic agent compound in any dermal formulation described herein is about 10:1, about 8:1, about 6:1, about 5:1, about 4:1, about 2:1, about 1:1, about 1:2, about 1:4, about 1:5, about 1:6, about 1:8, or about 1:10.

In some embodiments, the DP₂ receptor antagonist compound and the additional therapeutic agent are in the same pharmaceutical composition. In some embodiments, the DP₂ receptor antagonist compound and the additional therapeutic agent are in separate pharmaceutical compositions. In some embodiments, the DP₂ receptor antagonist compound and the additional therapeutic agent are in separate pharmaceutical compositions wherein the DP₂ receptor antagonist compound is administered topically and the additional therapeutic agent is administered by the same route or by a different route (e.g. oral administration). In some embodiments, the DP₂ receptor antagonist compound and the additional therapeutic agent are administered at the same time. In some embodiments, the DP₂ receptor antagonist compound and the additional therapeutic agent are administered at different times.

### Further Forms of Compounds

In some embodiments, the therapeutic agent(s) (e.g. DP₂ receptor antagonist and/or second therapeutic agent) is present in the pharmaceutical composition as a pharmaceutically acceptable salt. In some embodiments, pharmaceutically acceptable salts are obtained by reacting the therapeutic agent(s) with an acid. In some other embodiments, pharmaceutically acceptable salts are obtained by reacting the therapeutic agent(s) with a base. In some embodiments, the therapeutic agents are used as pharmaceutically acceptable salts in the preparation of the pharmaceutical compositions described herein. In other embodiments, the therapeutic agents are used as free-acid or free-base form in the manufacture of the pharmaceutical compositions described herein. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, the therapeutic agent(s) is/are reacted with an organic base, such as, but not limited to, ethanolamine, diethanolamine, triethanolamine, tromethamine, N-methylglucamine, dicyclohexylamine, tris(hydroxymethyl)methylamine. In other cases, the therapeutic agent(s) form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like. In some embodiments, a pharmaceutically acceptable salt of a compound disclosed herein is a sodium salt.

In some embodiments, the therapeutic agents disclosed herein possess one or more stereocenters and each center exists independently in either the R or S configuration. The compounds presented herein include all diastereomeric, enantiomeric, and epimeric forms as well as the appropriate mixtures thereof.

In some embodiments, sites on the therapeutics agents disclosed herein are susceptible to various metabolic reactions Therefore incorporation of appropriate substituents at the places of metabolic reactions will reduce, minimize or eliminate the metabolic pathways. In specific embodiments, the appropriate substituent to decrease or eliminate the susceptibility of the aromatic ring to metabolic reactions is, by way of example only, a halogen, deuterium or an alkyl group.

In some embodiments, the compounds described herein are labeled isotopically (e.g. with a radioisotope) or by another other means, including, but not limited to, the use of chromophores or fluorescent moieties, bioluminescent labels, or chemiluminescent labels. In some embodiments, compounds described herein are isotopically-labeled, which are identical to those recited in the various formulae and structures presented herein, but for the fact that one or more atoms are replaced by an atom having an atomic mass or mass number different from the atomic mass or mass number usually found in nature. In some embodiments, one or more hydrogen atoms are replaced with deuterium. In some embodiments, metabolic sites on the compounds described herein are deuterated. In some embodiments, substitution with deuterium affords certain therapeutic advantages resulting from greater metabolic stability, such as, for example, increased in vivo half-life or reduced dosage requirements.

### Certain Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. In this application, the use of “or” or “and” means “and/or” unless stated otherwise. Furthermore, use of the term “including” as well as other forms, such as “include”, “includes,” and “included,” is not limiting.

“Alkoxy” refers to (alkyl)O—, where alkyl is as defined herein.

“Alkyl” refers to an aliphatic hydrocarbon group. The alkyl may be saturated or unsaturated. In one aspect, alkyl groups are selected from methyl, ethyl, propyl, iso-propyl, n-butyl, iso-butyl, sec-butyl, and t-butyl.

“Cycloalkyl” refers to a monocyclic aliphatic, non-aromatic radical, wherein each of the atoms forming the ring (i.e. skeletal atoms) is a carbon atom. Cycloalkyl groups include, for example, cyclopropyl, cyclobutyl, cyclopentyl, and cyclohexyl.

“Halo”, “halogen” or “halide” means fluoro, chloro, bromo or iodo.

“Fluoroalkyl” refers to an alkyl in which one or more hydrogen atoms are replaced by a fluorine atom. In one aspect, a fluoroalkyl is selected from —CF₃, —CHF₂, —CH₂F, —CH₂CF₃ and —CF₂CF₃.

“Fluoroalkoxy” refers to (fluoroalkyl)O—, where fluoroalkyl is as defined herein.

“Heteroalkyl” refers to an alkyl group in which one or more skeletal atoms of the alkyl are selected from an atom other than carbon, e.g., oxygen, nitrogen (e.g. NH or Nalkyl), sulfur, or combinations thereof. In one aspect, heteroalkyl refers to an alkyl group in which one of the skeletal atoms of the alkyl is oxygen, nitrogen, or sulfur. In another aspect, heteroalkyl refers to an alkyl group in which one of the skeletal atoms of the alkyl is oxygen.

“6-Membered heteroaryl” includes pyridinyl, pyridazinyl, pyrimidinyl, pyrazinyl and triazinyl.

“Aryl” refers to phenyl or naphthalenyl. In some embodiments, an aryl is a phenyl.

The term “haloalkyl” refers to an alkyl group in which one or more hydrogen atoms are replaced by one or more halide atoms. In one aspect, a haloalkyl is a C₁-C₄haloalkyl.

The terms “heteroaryl” or, alternatively, “heteroaromatic” refers to an aryl group that includes one or more ring heteroatoms selected from nitrogen, oxygen and sulfur. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. In one aspect, the heteroaryl is a C₁-C₁₀heteroaryl. In another aspect, the heteroaryl is a C₂-C₉heteroaryl. In some cases, the heteroaryl includes at least one N atom in the ring. In some cases, the heteroaryl includes 1 or 2 N atom in the ring. In some cases, the heteroaryl includes 1 to 4 heteroatoms in the ring selected from O, N, and S. In one aspect, monocyclic heteroaryl is a C₁-C₅heteroaryl. In one aspect, bicyclic heteroaryl is a C₅-C₁₀heteroaryl.

A “heterocycloalkyl” or “heteroalicyclic” group refers to a cycloalkyl group that includes at least one heteroatom selected from nitrogen, oxygen and sulfur. Examples of heterocycloalkyl groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidino, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. In some embodiments, the heterocycloalkyl is selected from oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, and indolinyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl includes 1 or 2 heteroatoms in the ring selected from O, S, and N.

The term “optionally substituted” or “substituted” means that the referenced group may be substituted with one or more additional group(s) individually and independently selected from halogen, —OH, —CN, C₁-C₄alkyl, C₁-C₄fluoroalkyl, C₁-C₄alkoxy, C₁-C₄fluoroalkoxy, —NH₂, —NH(C₁-C₄alkyl), —N (C₁-C₄alkyl)₂, and C₁-C₄heteroalkyl. In some cases, substituted groups are substituted with one or more substituents selected from halogen, —OH, —OC₁-C₄alkyl, C₁-C₄alkyl, C₁-C₄heteroalkyl, C₁-C₄fluoroalkyl and —OC₁-C₄fluoroalkyl. For example, in some embodiments, a referenced substituted group is substituted with at least one group selected from halogen, —OH, —CN, —CH₃, —CH₂CH₃, —CF₃, —OCH₃, —OCH₂CH₃, and —OCF₃. In some cases, the referenced substituted group is substituted with 1 or 2 of the aforementioned groups.

“Prodrug” refers to an agent that is converted into the parent drug in vivo. In some situations, prodrugs are often useful because they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of carboxylic acid containing compound which is administered as an ester (the “prodrug”) and then is metabolically hydrolyzed to the carboxylic acid. In some embodiments, a prodrug is an alkyl ester prodrug. In some embodiments, a prodrug is a C₁-C₄alkyl ester prodrug. In some embodiments, a prodrug is a methyl ester or ethyl ester prodrug. A further example of a prodrug might be a short peptide (polyaminoacid) bonded to an acid group where the peptide is metabolized to reveal the active moiety. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they are easier to administer than the parent drug. In certain embodiments, the prodrug of a compound described herein is bioavailable by oral administration whereas the parent is not. Furthermore, in some embodiments, the prodrug of a compound described herein has improved solubility in pharmaceutical compositions over the parent drug. Prodrug forms of the herein described compounds, wherein the prodrug is metabolized in vivo to produce a derivative as set forth herein are included within the scope of the claims. Indeed, some of the herein-described compounds are a prodrug for another derivative or active compound.

The terms “individual,” “patient,” or “subject” are used interchangeably and refer to any mammal. In some embodiments, the mammal is a human. In some embodiments, the mammal is a non-human primate such as chimpanzee, and other apes and monkey species. In some embodiments, the mammal is a farm animal such as cattle, horse, sheep, goat, or swine. In some embodiments, the mammal is a domestic animal such as rabbit, dog, or cat. In some embodiments, the mammal is a laboratory animal, including rodents, such as rats, mice and guinea pigs, and the like.

The terms “treat,” “treating” or “treatment,” and other grammatical equivalents as used herein, include alleviating, abating, inhibiting, reducing, ameliorating, delaying the onset of, arresting the progression of, and/or inducing the regression of a disorder, disease or condition and/or the symptoms of a disorder, disease or condition. The terms also include prophylactic treatment of a disease or condition. The terms further include achieving any therapeutic benefit. Therapeutic benefit means the eradication or amelioration of the underlying disorder or disease or condition being treated, and/or the eradication or amelioration of one or more of the physiological symptoms associated with the underlying disorder or disease or condition such that an improvement is observed in the individual.

The terms “prevent,” “preventing” or “prevention,” and other grammatical equivalents as used herein include inhibiting (arresting or stopping) the development of a disorder, disease or condition, and/or inhibiting (arresting or stopping) the further progression of a disorder, disease or condition. These terms are intended to include prophylaxis. For prophylactic benefit, the compositions are administered to an individual at risk of developing a particular disorder, disease or condition, or to an individual reporting one or more of the physiological symptoms of a disorder, disease or condition, or to an individual at risk of reoccurrence of the disorder, disease or condition.

The terms “effective amount” or “therapeutically effective amount” as used herein, refer to an amount of an agent (e.g. DP₂ receptor antagonist compound) being administered which achieves a desired result, e.g., to relieve to some extent one or more symptoms of a disease, disorder or condition being treated. In certain instances, the result is a reduction and/or alleviation of at least one sign, symptom, or cause of a disorder, disease or condition, or any other desired alteration of a biological system.

### Topical Formulations

In some embodiments, a topical formulation disclosed herein facilitates the delivery of a DP₂ receptor antagonist compound to the skin for a local effect (i.e., an effect that is limited to the skin). In certain instances, local administration of a DP₂ receptor antagonist compound reduces or eliminates side-effects that are associated with systemic administration of a DP₂ receptor antagonist compound.

Topical formulations include, but are not limited to, ointments, creams, lotions, solutions, pastes, gels, sticks, liposomes, nanoparticles, patches, bandages and wound dressings.

### Creams and Lotions

Disclosed herein, in certain embodiments, is a topical formulation of a DP₂ receptor antagonist compound wherein the topical formulation is in the form of a cream. In certain instances, creams are semisolid (e.g., soft solid or thick liquid) formulations that include a DP₂ receptor antagonist compound dispersed in an oil-in-water emulsion or a water-in-oil emulsion. Disclosed herein, in certain embodiments, is a topical formulation of a DP₂ receptor antagonist compound wherein the topical formulation is in the form of a lotion. In certain instances, lotions are fluid emulsions (e.g., oil-in-water emulsions or a water-in-oil emulsions). In some embodiments, the hydrophobic component of a lotion and/or cream is derived from an animal (e.g., lanolin, cod liver oil, and ambergris), plant (e.g., safflower oil, castor oil, coconut oil, cottonseed oil, menhaden oil, palm kernel oil, palm oil, peanut oil, soybean oil, rapeseed oil, linseed oil, rice bran oil, pine oil, sesame oil, or sunflower seed oil), or petroleum (e.g., mineral oil, or petroleum jelly).

In certain instances, lotions and creams have a “drying” effect on dermatological diseases or conditions (e.g., some or all fluid exuded from the disorder is miscible in the ointment) and are thus useful for dermatological diseases or conditions characterized by the exudation of fluids.

### Ointments

Disclosed herein, in certain embodiments, is a topical formulation of a DP₂ receptor antagonist compound wherein the topical formulation is in the form of an ointment. In certain instances, ointments are semisolid preparations that soften or melt at body temperature. In certain instances, ointments re-hydrate the skin and are thus useful for dermatological diseases or conditions characterized by loss of moisture.

### Pastes

Disclosed herein, in certain embodiments, is a topical formulation of a DP₂ receptor antagonist compound wherein the topical formulation is in the form of a paste. In certain instances, pastes contain at least 20% solids. In certain instances, pastes are ointments that do not flow at body temperature. In certain instances, pastes re-hydrate the skin and are thus useful for dermatological diseases or conditions characterized by loss of moisture. In certain instances, pastes serve as protective coatings over areas to which they are applied.

### Gels

Disclosed herein, in certain embodiments, is a topical formulation of a DP₂ receptor antagonist compound wherein the topical formulation is in the form of a gel. In certain instances, gels are semisolid (or semi-rigid) systems consisting of dispersions of large organic molecules dispersed in a liquid. In certain instances, gels are water-soluble and are removed using warm water or saline. In certain instances, gels re-hydrate the skin and are thus useful for dermatological diseases or conditions characterized by loss of moisture.

### Sticks

Disclosed herein, in certain embodiments, is a topical formulation of a DP₂ receptor antagonist compound wherein the topical formulation is in the form of a stick. In certain instances, sticks are solid dosage forms that melt at body temperature. In some embodiments, a stick comprises a wax, a polymer, a resin, dry solids fused into a firm mass, and/or fused crystals. In some embodiments, a topical formulation of a DP₂ receptor antagonist compound is in the form of a styptic pencil (i.e., a stick prepared by (1) heating crystals until they lose their water of crystallization and become molten, and (2) pouring the molten crystals into molds and allowing them to harden). In some embodiments, a topical formulation of a DP₂ receptor antagonist compound is in the form of stick wherein the stick comprises a wax (e.g., the wax is melted and poured into appropriate molds in which they solidify in stick form).

In some embodiments, a topical formulation of a DP₂ receptor antagonist compound is in the form of stick wherein the stick comprises a melting base (i.e., a base that softens at body temperature). Examples of melting bases include, but are not limited to, waxes, oils, polymers and gels. In some embodiments, a topical formulation of a DP₂ receptor antagonist compound is in the form of stick wherein the stick comprises a moisten base (i.e., a base that is activated by the addition of moisture).

### Patches

Disclosed herein, in certain embodiments, is a topical formulation of a DP₂ receptor antagonist compound wherein the topical formulation is administered via a patch. In some embodiments, a topical formulation disclosed herein is dissolved and/or dispersed in a polymer or an adhesive. In some embodiments, a patch disclosed herein is constructed for continuous, pulsatile, or on demand delivery of a DP₂ receptor antagonist compound.

### Wound Dressings

Disclosed herein, in certain embodiments, is a topical formulation of a DP₂ receptor antagonist compound wherein the topical formulation is administered with (or via) a wound dressing. Wound dressings include, but are not limited to gauzes, transparent film dressings, hydrogels, polyurethane foam dressings, hydrocolloids and alginates. In certain instances, wound dressings (1) maintain moisture in the wound, (2) are semipermeable, (3) are semiocclusive, (4) allow for autolytic debridement, (5) protect from external contaminants, (6) absorb exuded fluids, and/or (7) allow for wound visualization.

### Dermatological Excipients

Disclosed herein, in certain embodiments, is a topical formulation of a DP₂ receptor antagonist compound wherein the topical formulation comprises a penetration enhancer. Penetration enhancers include, but are not limited to, hyaluronidase (e.g., PH-20), sodium lauryl sulfate, sodium laurate, polyoxyethylene-20-cetyl ether, laureth-9, sodium dodecylsulfate, dioctyl sodium sulfosuccinate, polyoxyethylene-9-lauryl ether (PLE), Tween 80, nonylphenoxypolyethylene (NP-POE), polysorbates, sodium glycocholate, sodium deoxycholate, sodium taurocholate, sodium taurodihydrofusidate, sodium glycodihydrofusidate, oleic acid, caprylic acid, mono- and di-glycerides, lauric acids, acylcholines, caprylic acids, acylcarnitines, sodium caprates, EDTA, citric acid, salicylates, DMSO, decylmethyl sulfoxide, ethanol, isopropanol, propylene glycol, polyethylene glycol, glycerol, propanediol, diethylene glycol monoethyl ether, and alkyl glycosides (e.g., dodecyl maltoside).

Disclosed herein, in certain embodiments, is a topical formulation of a DP₂ receptor antagonist compound wherein the topical formulation comprises a gelling (or thickening) agent. In some embodiments, a topical formulation disclosed herein further comprises from about 0.1% to about 5%, more preferably from about 0.1% to about 3%, and most preferably from about 0.25% to about 2%, of a gelling agent. In certain embodiments, the viscosity of a topical formulation disclosed herein is in the range from about 100 to about 500,000 cP, about 100 cP to about 1,000 cP, about 500 cP to about 1500 cP, about 1000 cP to about 3000 cP, about 2000 cP to about 8,000 cP, about 4,000 cP to about 10,000 cP, about 10,000 cP to about 50,000 cP.

Suitable gelling agents for use in preparation of the gel topical formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, acacia (gum arabic), agar, aluminum magnesium silicate, sodium alginate, sodium stearate, bladderwrack, bentonite, carbomer, carrageenan, carbopol, xanthan, cellulose, microcrystalline cellulose (MCC), ceratonia, chondrus, dextrose, furcellaran, gelatin, ghatti gum, guar gum, hectorite, lactose, sucrose, maltodextrin, mannitol, sorbitol, honey, maize starch, wheat starch, rice starch, potato starch, gelatin, sterculia gum, polyethylene glycol (e.g. PEG 200-4500), gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), or combinations thereof.

Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

Gels can also be classified as being hydrophobic or hydrophilic. In certain embodiments, the base of a hydrophobic gel consists of a liquid paraffin with polyethylene or fatty oils gelled with colloidal silica, or aluminum or zinc soaps. In contrast, the base of hydrophobic gels usually consists of water, glycerol, or propylene glycol gelled with a suitable gelling agent (e.g., tragacanth, starch, cellulose derivatives, carboxyvinylpolymers, and magnesium-aluminum silicates).

Suitable agents for use in fomulations that are applied as liquids and gel upon application to the skin into a film include but are not limited to polymers composed of polyoxypropylene and polyoxyethylene that are known to form thermoreversible gels when incorporated into aqueous solutions. These polymers have the ability to change from the liquid state to the gel state at temperatures close to body temperature, therefore allowing useful formulations that are applied as gels and/or films to the affected area. Examples of polymers that gel at body temperature and are used in gels and/or films described herein include and are not limited to poloxamers (e.g., PLURONICS F68®, F88®, F108®, and F127®, which are block copolymers of ethylene oxide and propylene oxide). The liquid state-to-gel state phase transition is dependent on the polymer concentration and the ingredients in the solution.

In some embodiments, the formulations and compositions disclosed herein are administered as a dermal paint. As used herein, paints (also known as film formers) are solutions comprised of a solvent, a monomer or polymer, an active agent, and optionally one or more pharmaceutically-acceptable excipients. After application to a tissue, the solvent evaporates leaving behind a thin coating comprised of the monomers or polymers, and the active agent. The coating protects active agents and maintains them in an immobilized state at the site of application. This decreases the amount of active agent which may be lost and correspondingly increases the amount delivered to the affected area of the skin of an individual. By way of non-limiting example, paints include collodions (e.g. Flexible Collodion, USP), and solutions comprising saccharide siloxane copolymers and a cross-linking agent. Collodions are ethyl ether/ethanol solutions containing pyroxylin (a nitrocellulose). After application, the ethyl ether/ethanol solution evaporates leaving behind a thin film of pyroxylin. In solutions comprising saccharide siloxane copolymers, the saccharide siloxane copolymers form the coating after evaporation of the solvent initiates the cross-linking of the saccharide siloxane copolymers.

In some instances, the topical formulations described herein comprise pressure sensitive adhesives (e.g., polyalkyloxazoline polymers) and allow for application of an adhesive film to an affected area of skin.

Disclosed herein, in certain embodiments, is a topical formulation of a DP₂ receptor antagonist compound wherein the topical formulation comprises an emollient. Emollients include, but are not limited to, castor oil esters, cocoa butter esters, safflower oil esters, cottonseed oil esters, corn oil esters, olive oil esters, cod liver oil esters, almond oil esters, avocado oil esters, palm oil esters, sesame oil esters, squalene esters, kikui oil esters, soybean oil esters, acetylated monoglycerides, ethoxylated glyceryl monostearate, hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate, oleyl myristate, oleyl stearate, and oleyl oleate, pelargonic acid, lauric acid, myristic acid, palmitic acid, stearic acid, isostearic acid, hydroxystearic acid, oleic acid, linoleic acid, ricinoleic acid, arachidic acid, behenic acid, erucic acid, lauryl alcohol, myristyl alcohol, cetyl alcohol, hexadecyl alcohol, stearyl alcohol, isostearyl alcohol, hydroxystearyl alcohol, oleyl alcohol, ricinoleyl alcohol, behenyl alcohol, erucyl alcohol, 2-octyl dodecanyl alcohol, lanolin and lanolin derivatives, beeswax, spermaceti, myristyl myristate, stearyl stearate, carnauba wax, candelilla wax, lecithin, and cholesterol.

Disclosed herein, in certain embodiments, is a topical formulation of a DP₂ receptor antagonist compound wherein the topical formulation comprises abrasives, absorbents, anticaking agents, astringents, essential oils, fragrances, skin-conditioning agents, skin healing agents, skin protectants (e.g., sunscreens, or ultraviolet light absorbers or scattering agents), skin soothing agents, or combinations thereof.

Pharmaceutical topical formulations disclosed herein are formulated in any suitable manner. Any suitable technique, carrier, and/or excipient is contemplated for use with the DP₂ receptor antagonist compounds disclosed herein. For a summary of pharmaceutical topical formulations described herein see Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Eighth Ed. (Lippincott Williams & Wilkins 2004), Muller, R. H. et al. Advanced Drug Delivery Reviews 59 (2007) 522-530, which are herein incorporated by reference for such disclosures.

In some embodiments, any dermal formulation described herein comprises between about 0.1 to about 50%, between about 0.1 to about 25%, between about 0.1 to about 10%, between about 0.1 to about 5%, or between about 0.1 to about 1% of a DP₂ receptor antagonist by weight of the formulation.

### Dosing

Disclosed herein, in certain embodiments, is a topical formulation of a DP₂ receptor antagonist compound wherein the topical formulation administered for prophylactic and/or therapeutic treatments. In certain instances, amounts effective for this use will depend on the severity and course of the disease, disorder or condition, previous therapy, the individual's health status and response to the drugs, and the judgment of the treating physician.

In some embodiments, where a dermatological disease or condition does not improve, a topical formulation disclosed herein is administered chronically (i.e., for an extended period of time, including throughout the duration of the individual's life). In some embodiments, where a dermatological disease or condition does improve, a topical formulation disclosed herein is given continuously; alternatively, the dose of active agent being administered is temporarily reduced or temporarily suspended for a certain length of time (i.e., a “drug holiday”). In some embodiments, a drug holiday lasts between 2 days and 1 year, including all integers in between. In some embodiments, the dose reduction during a drug holiday is from about 10% to about 100%, including all integers in between.

In some embodiments, where a dermatological disease or condition does improve, a topical formulation disclosed herein is administered as a maintenance dose. In some embodiments, where a dermatological disease or condition does improve, a topical formulation disclosed herein is administered with reduced frequency or at a reduced dose.

In one embodiment, a topical formulation disclosed herein is formulated for controlled release of a DP₂ receptor antagonist compound. In some embodiments, a DP₂ receptor antagonist compound is released over a time period exceeding 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

### EXAMPLES

The following examples are illustrative and non-limiting to the scope of the formulations and methods described herein.

### Example 1

### Topical Formulation of a DP₂ Receptor Antagonist Compound

A topical formulation of a DP₂ receptor antagonist compound is prepared by mixing a DP₂ receptor antagonist compound with propylene glycol, transcutol and water. In one aspect, the topical formulation includes a DP₂ receptor antagonist compound (10 mg/mL) in a solution of 75% propylene glycol, 15% transcutol, 10% water.

### Example 2

### Lotion Formulation of DP₂ Receptor Antagonist Compound

A DP₂ receptor antagonist compound (15 g) is mixed ethanol. Tween 80 (5 mL) is added. Carbopol 974 is dispersed in 1 L water and the ethanol mixture is slowly added to the aqueous mixture. The mixture is stirred and the volume is adjusted to 1500 mL with purified water.

### Example 3

### Hydrogen Formulation of DP₂ Receptor Antagonist Compound

DP₂ receptor antagonist compound (150 g), benzyl alcohol (40 mL), and glycerin are added to about 3200 mL of purified water. Slowly add Pluronic F127 (45 g) into the mixture. Adjust pH to 7.0 with phosphate buffer. Add purified water to bring the volume to 4000 mL. Finally, add trolamine (dropwise) until a gel is formed.

### Example 4

### Stick Formulation of DP₂ Receptor Antagonist Compound

A mixture of melted beeswax (300 g), cocoa butter (50 g), paraffin (125 g) and lanolin (50 g) is added to a mixture of DP₂ receptor antagonist compound (50 g) and petrolatum (180 g). The mixture is stirred for 40 minutes and poured into molds.

### Example 5

### DP₂/CRTH2 Binding Assay

The ability of a compound to bind to the human DP₂ receptor is assessed via a radioligand binding assay using [³H]PGD₂. HEK293 cells stably expressing recombinant human DP₂ are resuspended in 10 mM Hepes, 7.4 containing 1 mM DTT, lysed and centrifuged at 75,000×g to pellet the membranes. The membranes are resuspended in 10 mM Hepes, 7.4 containing 1 mM DTT and 10% glycerol to approximately 5 mg protein/ml. Membranes (2-10 μg protein/well) are incubated in 96-well plates with 1 nM [³H]PGD₂ and test compound in Assay Buffer (50 mM Hepes, 10 mM MnCl₂, 1 mM EDTA, plus or minus 0.2% human serum albumin, pH 7.4) for 60 minutes at room temperature. The reactions are terminated by rapid filtration through Whatman GF/C glass fibre filter plates. The filter plates were pre-soaked in 0.33% polythylenimine for 30 minutes at room temperature then washed in Wash Buffer (50 mM Hepes, 0.5 M NaCl pH 7.4) prior to harvesting. After harvesting, the filter plates are washed 3 times with 1 ml cold Wash Buffer then dried. Scintillant is then added to the plates and the radioactivity retained on the filters is determined on a Packard TopCount (Perkin Elmer). Specific binding is determined as total radioactive binding minus non-specific binding in the presence of 10 μM PGD₂. IC₅₀s were determined using GraphPad prism analysis of drug titration curves.

### Example 6

### DP₂ Binding Assay

The ability of a compound to bind to the human DP1 receptor was evaluated via a radioligand membrane binding assay using the DP₁ selective synthetic ligand [³H]BWA868C. Packed human platelets (Biological Specialty Corporation), were resuspended in 6 volumes of Hepes/HBSS buffer (10 mM Hepes, 1 mM DTT in Hanks Balanced Salt Solution (HBSS)), lysed and centrifuged at 75,000×g to pellet the membranes. Membranes were resuspended in Hepes/HBSS buffer to approximately 12 mg protein/ml. Membranes (20 μg protein/well) are incubated in 96-well plates with 2 nM [³H]BWA868C and test compound in Assay Buffer (50 mM Hepes, 10 mM MnCl₂, 1 mM EDTA, plus or minus 0.2% human serum albumin, pH 7.4) for 60 minutes at room temperature. The reactions are terminated by rapid filtration through Whatman GF/C glass fibre filter plates. The filter plates were pre-soaked in 0.33% polethylenimine for 30 minutes at room temperature then washed in Wash Buffer (50 mM Hepes, 0.5 M NaCl pH 7.4) prior to harvesting. After harvesting, the filter plates are washed 3 times with 1 ml cold Wash Buffer then dried. Scintillant is then added to the plates and the radioactivity retained on the filters is determined on a Packard TopCount (Perkin Elmer). Specific binding is determined as total radioactive binding minus non-specific binding in the presence of 10 μM BW A868C. IC₅₀s were determined using GraphPad prism analysis of drug titration curves.

Representative data for compounds tested in Example 5 and Example 6 is presented in the following table.

TABLE 1
Representative Biological Data

| | hDP2 | hDP1 |
|---|---|---|
| Compound | μM | μM |
| [2′-(3-Benzyl-1-ethyl-ureidomethyl)-6-methoxy-4′- | A | C |
| trifluoromethyl-biphenyl-3-yl]-acetic acid | | |
| {3-[2-tert-Butylsulfanylmethyl-4-(2,2-dimethyl- | A | C |
| propionylamino)-phenoxy]-4-methoxy-phenyl}-acetic acid | | |
| {2′-[(N-cyclopropanecarbonyl-N-ethyl-amino)-methyl]- | A | C |
| 6-methoxy-4′-trifluoromethyl-biphenyl-3-yl}-acetic acid | | |
| [2′-[(N-cyclopropanecarbonyl-N-ethyl-amino)-methyl]-4′- | A | C |
| (6-ethoxy-pyridin-3-yl)-6-methoxy-biphenyl-3-yl]-acetic | | |
| acid | | |
| (5-{2-[(N-benzyloxycarbonyl-N-ethyl-amino)-methyl]-4- | A | C |
| trifluoromethyl-phenyl}-pyridin-3-yl)-acetic acid | | |
| {8-[(4-fluoro-benzenesulfonyl)-methyl-amino]-6,7,8,9- | A | C |
| tetrahydro-pyrido[3,2-b]indol-5-yl}-acetic acid | | |
| Ramatroban | B | C |

A = less than 0.3 μM;
B = greater than 0.3 μM but less than 1 μM;
C = greater than 1 μM.

### Example 7

### Rabbit Wound Healing and Hypertrophic Scar Model

Following anesthesia, ear wounds are created in 10 young adult female New Zealand rabbits, 4 wounds per ear on each ear for a total of 8 wounds per animal. Wounds were created using a 7-mm biopsy punch with the wound created to go to bare cartilage. A dissecting microscope is used to ensure complete removal of the epidermis, dermis and perichondrium in each wound. For the hypertrophic scar model, it is the removal of the perichondrial layer and subsequent delay in reepithelialization of the defect that results in the elevated scar. Each wound heals independently and is considered a separate sample.

Two treatment groups are examined to study the early phase and a later phase of wound healing. The early treatment group (n=15 rabbits, 120 wounds) are treated with either the test compound formulated as a 0.05-1.5% by weight topical formulation (solution, cream, ointment or gel) or placebo using the topical vehicle formulation post-wounding on days 0, 1, 2, 3, 4, 5, 6 and 7 and harvested on day 28 after wounding. The later treatment group (n=15 rabbits, 120 wounds) are treated with either the test compound formulated as a 0.05-1.5% topical formulation (solution, cream, ointment or gel) or placebo using the topical vehicle formulation post-wounding on days 7, 8, 9, 10, 11, 12, 13 and 14 and harvested on day 28 after wounding. Half of the wounds in each group are treated with active compound and half are treated with placebo. Each wound is covered with a sterile dressing (Tegaderm; 3M) and dressings are changed daily following each treatment and as needed until the wound appears reepithelialized on gross examination. Wounds are excluded from analysis if there is evidence of infection, desiccation or necrosis.

At the end of each study wounds are harvested with a 5-mm margin of surrounding unwounded tissue. The scars are bisected and half of each wound is fixed in 4% neutral-buffered formaldehyde, dehydrated, embedded in paraffin, cut in 4-μm sections, and stained with Masson's trichrome or sirrus red. The other half of each wound is flash frozen in liquid nitrogen and stored for RNA extraction

### Histologic Analysis

Light microscopy is used to examine each tissue section and the degree of wound healing and scar hypertrophy are measured with a calibrated lens reticle in a blinded fashion. Wound healing parameters: Relevant measurements are granulation tissue ingrowth volume and height, wound epithelialization, and wound closure. Each parameter is assessed twice and the results are averaged.

Scar hypertrophy parameters: The scar elevation index is determined as described by Lu et al, J. Am. Coll. Surg., 2005, 201, p 391-397. The values are determined twice by in a blinded fashion and the results averaged.

### Example 8

### Rat Abraded Skin Model to Test for Acute Irritancy and Skin Penetration Following Dermal Administration

Male Sprague Dawley Rats (n=3 per group) are shaved on the back such that approximately 20% of the body area surface is exposed. The shaved skin is mildly abraded then 2 mL of a 10 mg/mL solution of the test compound in a formulation containing 75% propylene glycol, 15% transcutol, 10% water is applied to the abraded skin. The site of administration is occluded for 24 hours after which time the skin is cleansed of excess material (if any). Plasma samples are drawn from each rat at 2, 4, 24 and 72 hours.

Visual analysis of erythema, edema or other dermal findings are recorded prior to dosing then at 24, 48 and 72 hours. Scoring follows a Draize protocol. A Draize score of zero indicates no irritancy of the formulation. The plasma levels of the compounds are recorded at 24 h.

### Example 9

### Effects of DP2 Antagonist on Mucin Levels

BALB/c mice were divided into groups and acclimatized in cages for 24 hours (day 0). The control group was exposed to air and the test group was exposed to smoke from seven unfiltered cigarettes per day for 8 days (day 1 to day 8). Mice were divided into groups and are administered compound starting at day 1 and up to day 13. On day 14, bronchoalveolar lavage fluid (BALF) is tested for influx of cells, cytokines, chemokines (e.g., KC, MIP-2, IL-6), mucin, and/or proteins. Lung histology is also examined. One group received treatment with DP₂ receptor antagonist 5-{2-[(N-Benzyloxycarbonyl-N-ethyl-amino)-methyl]-4-trifluoromethyl-phenyl}-pyridin-3-yl)-acetic acid (Compound 1; 10 mg/kg qd), a second group received treatment with FLAP inhibitor compound H (3-(3-(tert-butylthio)-1-(4-(6-methoxypyridin-3-yl)benzyl)-5-((5-methylpyridin-2-yl)methoxy)-1H-indol-2-yl)-2,2-dimethylpropanoic acid) (30 mg/kg, b.i.d.); and the third group received treatment with a combination of DP₂ receptor antagonist Compound 1 (10 mg/kg qd) and FLAP inhibitor compound H (30 mg/kg, b.i.d.).

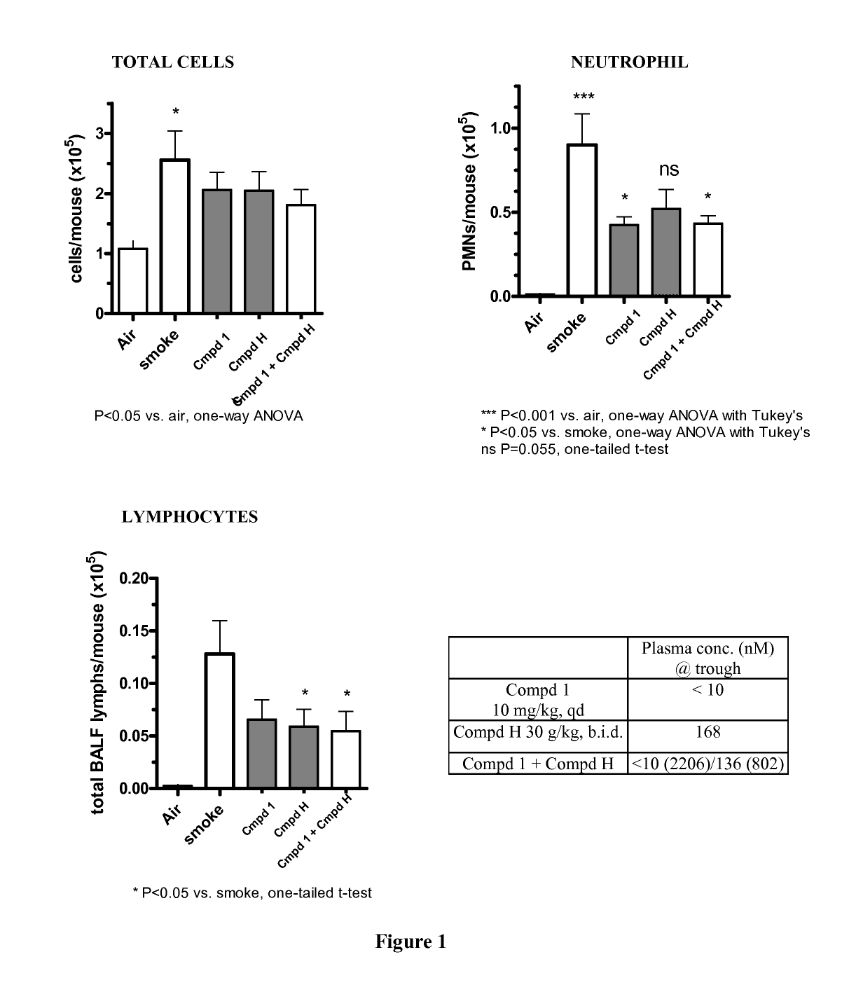

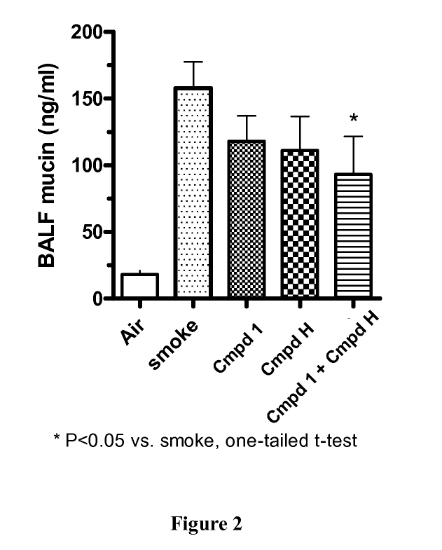

FIG. 1 illustrates the effect of DP₂ receptor antagonism and FLAP inhibition on the number of total cells, neutrophils and lymhocytes present in BALF. FIG. 2 illustrates the effect of a combination of a DP₂ receptor antagonist and a FLAP inhibitor on the presence of mucin in BALF. In the subchronic smoking mouse model, the effects of a combination of a DP₂ receptor antagonist compound and a FLAP inhibitor compound on mucin secretion in BALF were additive, i.e., a combination of Compound 1 and Compound H reduced the amount of mucin in BALF more than each compound alone. In one aspect, the effects of topical administration of a DP₂ receptor antagonist, either alone or in combination with a FLAP inhibitor compound, to the skin has similar effects (e.g., is mechanistically expected to) in dermal tissues as observed in the BALF.

### Example 10

### Mouse Epicutaneous Sensitization Model

Using the procedure described in Boehme et al, Internat. Immunol., 2009, 21(1), p1-17 mice are anaesthetized and the dorsal skin shaved. Gauze (1×1 cm²) soaked in either PBS or 1% ovalbumin (Fraction VI) solution in PBS is placed on the exposed skin surface. The gauze is held in place using a bioocclusive dressing (e.g. IV3000 MPV transparent dressing) and after 3 days the gauze is replaced with a fresh gauze patch kept in place for 4 days such that the total sensitization period is 7 days. The mice have a 2 week interval, then the 7 day patch protocol is repeated followed by a second 2 week interval and then a third 7 day patch protocol is carried out. One day following the last of the three sensitizations (day 50) the mice are sacrificed, serum and patched skin collected for analysis. Mice are dosed orally either with vehicle or with a DP2 antagonist compound in vehicle daily during the second and third sensitization periods. The patched skin is examined by histology, mRNA and for cytokine and chemokine levels.

### Example 11

### Effect of a DP₂ Receptor Antagonist Compound on the Treatment of Canine Atopic Dermatitis

The protocol described in Nuttall et al, Vet Dermatol., 2009, 20(3), 191-198 was followed with modifications. Dogs with a canine atopic dermatitis (AD) extent and severity index (CADESI-03)> or =50 are randomly allocated to receive either vehicle solution or drug solution (n=15 each group) as a spray. Animals receive two sprays from 10 cm distance once a day for 28 days. The area treated is 100 cm². CADESI score, pruritis and owner satisfaction (using a 5 point scale) is evaluated at day 28. Haematology and drug plasma concentrations are measured at baseline and at day 7, day 14, day 21, and day 28.

### Example 12

### Effect of a DP₂ Receptor Antagonist Compound on the Treatment of Atopic Dermatitis

This is a double-blind, placebo-controlled, randomized study to evaluate the safety, efficacy and tolerability of a topically applied DP₂ receptor antagonist in the treatment of subjects with atopic dermatitis.

Subjects are to read and sign an informed consent agreement and must be willing to comply with the protocol. Exclusion criteria include a diagnosis of an acute systemic illness; suspected viral, fungal or bacterial infection of the skin; severe hepatic disease or renal impairment; systemic treatment within the preceding thirty days and topical treatment within the preceding 30 days. Pregnant or lactating women are excluded and women of child-bearing potential must have a documented negative urine pregnancy test and must be practicing a medically proven form of contraception during the course of the study.

Two hundred patients with clinically diagnosed atopic dermatitis based on the evaluation of seven disease signs are randomly allocated to a placebo group and a drug-treated group (randomized 5:3 to drug treatment:placebo). The seven disease signs (infiltration, scaling, erythema, lichenification, vesicles, papules, and excoriation) are each given a ranking using a four-point scale: absent (0), mild (1), moderate (2) and severe (3). The disease severity is determined by the total score for the seven clinical signs with a score ≧6 being required for study enrollment. The drug treated group receive a dermal administration of a DP₂ receptor antagonist compound formulated to an appropriate concentration of between 0.05 to 1.5% by weight in a clinically acceptable and safe topical formulation (solution, cream, ointment or gel). The placebo group receive the same topical formulation absent the active drug. Patients receive treatment once daily for 14 days with the treatment areas covering approximately 15% of the body surface on the basis of location, extent of involvement and severity of signs. Treatment is excluded on the face, hands, groin or axillae. Follow up study visits are scheduled for days 3, 7 and 14.

### Clinical Assessment

The seven disease signs (infiltration, scaling, erythema, lichenification, vesicles, papules, and excoriation) are each given a ranking using the four-point scale. The primary efficacy evaluation is based on the change from the baseline in the total severity score measured on days 3, 7 and 14. The efficacy of the drug treatment is also assessed by the investigator at day 14 based on overall improvement using the following scale: cleared (1), excellent (2), good (3), fair (4), poor (5), no effect (6), exacerbated (7). Patients also provide a self assessment on the final visit answering questions on: ease of application, lack of odor, and feeling on skin. Patients and investigators individually also provide an assessment of efficacy based on: excellent (1), good (2), fair (3), or poor (4).

The examples and embodiments described herein are for illustrative purposes and various modifications or changes suggested to persons skilled in the art are to be included within the spirit and purview of this application and scope of the appended claims. The section headings used herein are for organizational purposes only and are not to be construed as limiting the subject matter described.

1. A topical formulation comprising a DP₂ receptor antagonist and at least one pharmaceutically acceptable excipient to provide an ointment, cream, lotion, paste, gel, stick, a film, a patch or wound dressing, wherein the topical formulation is suitable for administration to the skin of a mammal.

2. The topical formulation of claim 1, wherein the DP₂ antagonist is a compound having the structure of Formula (I), pharmaceutically acceptable salt, pharmaceutically acceptable solvates, or prodrug thereof: 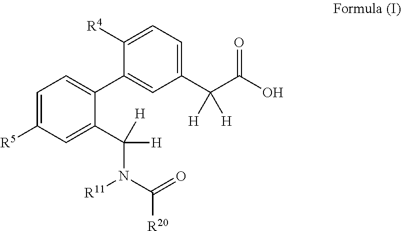
wherein,
R⁴ is H, halogen, —CN, —OH, C₁-C₄alkoxy, C₁-C₄fluoroalkyl, C₁-C₄fluoroalkoxy, or C₁-C₄heteroalkyl;
R⁵ is H, halogen, —CN, —NO₂, —OH, —OR¹³, —SR¹², —S(═O)R¹², —S(═O)₂R¹², —NHS(═O)₂R¹², —C(═O)R12, —OC(═O)_{R}¹², —CO₂R¹³, —OCO₂R¹³, —CH(R¹³)₂, —N(R¹³)₂, —C(═O)N(R¹³)₂, —OC(═O)N(R¹³)₂, —NHC(═O)NH(R¹³), —NHC(═O)R¹², —NHC(═O)OR¹², —C(OH)(R¹³)₂, —C₁-C₆alkyl, C₁-C₆fluoroalkyl, C₁-C₆fluoroalkoxy, C₁-C₆alkoxy, or C₁-C₆heteroalkyl;
or R⁵ is C₃-C₁₀cycloalkyl, a substituted or unsubstituted C₂-C₁₀heterocycloalkyl, a substituted or unsubstituted phenyl, a substituted or unsubstituted naphthyl, a substituted or unsubstituted monocyclic heteroaryl, or a substituted or unsubstituted bicyclic heteroaryl, wherein if R⁵ is substituted, then R⁵ is substituted with 1 or 2 R²¹ groups;
R²⁰ is C₁-C₄alkyl, C₃-C₆cycloalkyl, —CH₂O—C₁-C₄alkyl, —CH₂O-(substituted or unsubstituted phenyl), —CH(CH₃)—O-(substituted or unsubstituted phenyl), —C(CH₃)₂—O-(substituted or unsubstituted phenyl), —CH₂OCH₂-(substituted or unsubstituted phenyl), —OC₁-C₄alkyl, —O—CH₂-(substituted or unsubstituted phenyl), —O—CH(CH₃)-(substituted or unsubstituted phenyl), —NR¹⁶C₁-C₄alkyl, —NR¹⁶—CH₂-(substituted or unsubstituted phenyl), or —NR¹⁶—CH(CH₃)-(substituted or unsubstituted phenyl), wherein if the phenyl of R²⁰ is substituted, then the phenyl is substituted with 1 or 2 R²¹ groups; each R²¹ is independently selected from halogen, —OH, —OC₁-C₄alkyl, C₁-C₄alkyl, and —CF₃;
R¹⁶ is H or C₁-C₄alkyl;
R¹¹ is C₁-C₄alkyl, C₁-C₄fluoroalkyl, or C₃-C₆cycloalkyl;
R¹² is C₁-C₄alkyl, C₁-C₄heteroalkyl, or C₁-C₄fluoroalkyl;
each R¹³ is independently selected from H, C₁-C₄alkyl, C₁-C₄heteroalkyl, and C₁-C₄fluoroalkyl.

3. The topical formulation of claim 1, wherein the DP₂ antagonist is ramatroban, AMG 009, AMG 853, Compound 14 of WO 09/085177, AZD1981, AZD8075, AZD5985, ARRY-005, ARRY-006, ARRY-063, ODC9101 (OC459), OC499, OC1768, OC2125, OC2184, QAV680, MLN6095, ACT-129968, ADC3680, SAR398171, 5555739, AP768, [2′-(3-Benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxyphenyl}-acetic acid, TM30642, TM30643, TM30089, TM27632, and TM3170, {2′-[(N-cyclopropanecarbonyl-N-ethyl-amino)-methyl]-6-methoxy-4′-trifluoromethyl-biphenyl-3-yl}-acetic acid, [2′-[(N-cyclopropanecarbonyl-N-ethyl-amino)-methyl]-4′-(6-ethoxy-pyridin-3-yl)-6-methoxy-biphenyl-3-yl]acetic acid, (5-{2-[(N-benzyloxycarbonyl-N-ethyl-amino)-methyl]-4-trifluoromethyl-phenyl}-pyridin-3-yl)-acetic acid, or {8-[(4-fluoro-benzenesulfonyl)-methyl-amino]-6,7,8,9-tetrahydro-pyrido[3,2-b]indol-5-yl}-acetic acid, or a pharmaceutically acceptable salt, pharmaceutically acceptable solvate, or prodrug thereof.

4. The topical formulation of claim 1, wherein the topical formulation is used in the treatment of a prostaglandin D₂-dependent or prostaglandin D₂-mediated dermal disease or condition in a mammal.

5. The topical formulation of claim 4, wherein the prostaglandin D₂-dependent or prostaglandin D₂-mediated dermal disease or condition is scarring, a burn, dermal mucinosis, an immune disease or condition affecting the skin; a dermal proliferative disease or condition; an inflammatory disease or condition; a mast cell mediated disease or condition; a Th2 lymphocyte mediated disease or condition; an infection or combinations thereof.

6. The topical formulation of claim 4, wherein the prostaglandin D₂-dependent or prostaglandin D₂-mediated dermal disease or condition is atopic dermatitis, allergic dermatitis, bullous disorders, collagenoses, psoriasis, psoriatic lesions, contact dermatitis, eczema, urticaria, rosacea, hypertrophic scarring, keloid scar formation, scleroderma, Folliculitis keloidalis nuchae, Kawasaki Disease, Sjogren-Larsso Syndrome, Grover's disease, a first degree burn, a second degree burn, a third degree burn, a fourth degree burn, cutaneous mucinosis, solar keratosis, squamous cell carcinoma or melanoma.

7. The topical formulation of claim 1, wherein the topical formulation is used in the treatment of dermatitis, eczema, psoriasis, or cutaneous mucinosis.

8. The topical formulation of claim 1, wherein the topical formulation is used for decreasing dermal mucin concentrations in a mammal.

9. The topical formulation of claim 1, wherein the topical formulation is used in the treatment or prevention of itching in a mammal, in the treatment or prevention of a rash in a mammal, in the treatment or prevention of skin inflammation in a mammal.

10. The topical formulation of claim 1, wherein the topical formulation is used in the treatment or prevention of blisters, redness, swelling, scabbing, scaling, or combinations thereof in a mammal.

11. The topical formulation of claim 1, further comprising a second therapeutic agent.

12. A method of treating a dermal disease or condition in a mammal comprising topically administering a DP₂ receptor antagonist to the skin of the mammal.

13. The method of claim 12, wherein the DP₂ receptor antagonist compound is in a form suitable for topical administration to the skin of a mammal.

14. The method of claim 12, wherein the dermal disease or condition is a prostaglandin D₂-dependent or prostaglandin D₂-mediated dermal disease or condition.

15. The method of claim 12, wherein the dermal disease or condition is scarring, a burn, dermal mucinosis, an immune disease or condition affecting the skin; a dermal proliferative disease or condition; an inflammatory disease or condition; a mast cell mediated disease or condition; a Th2 lymphocyte mediated disease or condition; an infection or combinations thereof.

16. The method of claim 12, wherein the dermal disease or condition is atopic dermatitis, allergic dermatitis, bullous disorders, collagenoses, psoriasis, psoriatic lesions, contact dermatitis, eczema, urticaria, rosacea, hypertrophic scarring, keloid scar formation, scleroderma, Folliculitis keloidalis nuchae, Kawasaki Disease, Sjogren-Larsso Syndrome, Grover's disease, a first degree burn, a second degree burn, a third degree burn, a fourth degree burn, cutaneous mucinosis, solar keratosis, squamous cell carcinoma or melanoma.

17. The method of claim 12, wherein the dermal disease or condition is dermatitis, eczema, psoriasis, or cutaneous mucinosis.

18. The method of claim 12, wherein the DP₂ receptor antagonist compound is used for decreasing dermal mucin concentrations in a mammal.

19. The method of claim 12, wherein the DP₂ receptor antagonist compound is used in treatment or prevention of itching in a mammal, in the treatment or prevention of a rash in a mammal, in the treatment or prevention of skin inflammation in a mammal.

20. The method of claim 12, wherein the DP₂ receptor antagonist compound is used in treatment or prevention of blisters, redness, swelling, scabbing, scaling, or combinations thereof in a mammal.

21. The method of claim 12, further comprising a second therapeutic agent.

22. The method of claim 21, wherein the second therapeutic agent is an antibiotic, anti-fungal agent, steroid anti-inflammatory agent, non-steroidal anti-inflammatory agent, antihistamine, antiviral agent, mast cell stabilizer, cyclosporine, or a leukotriene modulator.

23. The method of claim 21, wherein the second therapeutic agent is a leukotriene modulator selected from 5-lipoxygenase (5-LO) inhibitors, 5-lipoxygenase activating protein (FLAP) inhibitors, and leukotriene receptor antagonists.
